(19) United States
(12) Patent Application Publication
(10) Pub. No.: US 2023/0018956 A1
(43) Pub. Date: Jan. 19, 2023

(54) SYSTEM AND METHOD FOR ASSESSING A CANCER STATUS OF BIOLOGICAL TISSUE

(71) Applicants: POLYVALOR, LIMITED PARTNERSHIP, Montréal (CA); THE ROYAL INSTITUTION FOR THE ADVANCEMENT OF LEARNING/MCGILL UNIVERSITY, Montréal (CA)

(72) Inventors: Frédéric LEBLOND, Outremont (CA); Kevin PETRECCA, Verdun (CA); Michael JERMYN, Longueuil (CA)

(73) Assignees: POLYVALOR, LIMITED PARTNERSHIP, Montréal (CA); THE ROYAL INSTITUTION FOR THE ADVANCEMENT OF LEARNING/MCGILL UNIVERSITY, Montréal (CA)

(21) Appl. No.: 17/865,690

(22) Filed: Jul. 15, 2022

Related U.S. Application Data
(63) Continuation of application No. 15/288,725, filed on Oct. 7, 2016, now abandoned.
Application No. 15/288,725 is a continuation of application No. PCT/CA2015/050288, filed on Apr. 8, 2015.
(60) Provisional application No. 61/976,558, filed on Apr. 8, 2014.

(51) Int. Cl. A61B 5/00; G06K 9/62; G01N 33/574; A61B 5/06; G06V 20/69; G01N 21/65

(52) U.S. Cl. CPC A61B 5/7267; A61B 5/4842; G06K 9/6282; A61B 5/0075; A61B 5/4064; G01N 33/57407; A61B 5/064; G06V 20/698; G01N 21/65; G01N 33/574; G06K 9/6257; A61B 2576/026; G06V 2201/03; A61B 2090/306

(57) ABSTRACT

A method for assessing a cancer status of biological tissue includes the steps of: obtaining a Raman spectrum indicating a Raman spectroscopy response of the biological tissue, the Raman spectrum captured using a fiber-optic probe of a fiber-optic Raman spectroscopy system; inputting the Raman spectrum into a boosted tree classification algorithm of a computer program, and using the boosted tree classification algorithm for comparing, in real-time, the captured Raman spectrum to reference data and assessing the cancer status of the biological tissue based on said comparison, the reference data being previously determined based on a set of reference Raman spectra indicating Raman spectroscopy responses of reference biological tissues wherein each of the reference biological tissues is associated with a known cancer status; and generating a real-time output indicating the assessed cancer status of the biological tissue,

### CROSS-REFERENCE TO RELATED APPLICATIONS

This application is a continuation of U.S. patent application Ser. No. 15/288,725 filed on October 7, 2016, pending, which is a continuation of International Application No. PCTICA2015/050288 filed on Apr. 8, 2015, which claims priority to U.S. Provisional Application No. 61/976,558, filed on Apr. 8, 2014, the entire contents of which are incorporated herein by reference in their entireties.

### TECHNICAL FIELD

The present disclosure relates generally to the characterization of biological tissue, and more particularly, methods and systems used for assessing the cancer status of biological tissue.

### BACKGROUND

Most currently employed medical techniques for detecting and/or classifying unhealthy biological tissue (such as, but not limited to, detecting a malignant tumor within surrounding tissue) require imaging of the tissue to first be conducted. The imagine results are typically used by a physician to assist in the determination as to whether any unhealthy biological tissue is present in the scanned tissue and/or to subsequently plan an appropriate surgical intervention. Commonly used imaging techniques include magnetic resonance imaging (MRI), X-rays and computed tomography (CT) scans, for instance.

Although such imaging techniques are acceptable for many applications certain limitations nevertheless exist with respect to the use of pre-operatively obtained imaging results in cases involving soft tissue in general, and brain tissue in particular. Such limitations include inherent technological restrictions (e.g. limited resolution or sensitivity of a scanned image) and spatial discrepancies resulting from movement of the relevant soft tissue between the time the imaging is conducted and the time a subsequent surgery is performed. Even MRI images, which are relatively accurate in comparison with other forms of imaging, and which relied upon for many diagnostic purposes and for planning surgical inventions, currently have a resolution and/or sensitivity which can sometimes be insufficiently precise for accurate diagnosis of the unhealthy tissue based on the results of the imaging scan alone. This is particularly true in cases where the unhealthy biological tissue is more difficult to clearly identify, such as at the margins of a tumor for example. In certain cases, this inability to fully identify a totality of the unhealthy biological tissue can result in only a partial resection of the unhealthy tissues of the patient during the resulting surgery. This, in turn, may negatively impact the eventual prognosis of the patient.

Certain surgical interventions conducted to remove unhealthy tissue identified pre-operatively from a scanned image, such as the resection of a tumor for example, permit the surgeon to intraoperatively visually inspect the tissue in order to make a determination as to whether the tissue in question should be reselected. In the case of malignant tumors In general, and brain tumors in particular, it is often possible to identify the presence of a tumor from a pre-operative scan and for a surgeon to subsequent intraoperatively locate the main mass of the tumor during surgery. However, it can sometimes be much more difficult for a surgeon to accurately distinguish, intraoperatively, all unhealthy tissue by visual inspection alone. This is particularly true in regions of mixed healthy arid unhealthy tissue,, such as at the margins of a tumor, or in cases when the unhealthy tissue is less easily visually identifiable, even under a microscope. The successful removal of the entirety of the unhealthy tissue, such as the entirety of a malignant tumor, therefore often relies significantly on the expertise of the surgeon in making such a determination intraoperatively. As complete resection of all cancerous tissue present is often directly linked to the rate of recovery and/or prognosis of the patient, much relies on the skill and expertise of the surgeon in intraoperatively evaluating and identifying all unhealthy tissue present, so that it can be reselected. In certain applications, such as with brain tumors, it is particularly undesirable to remove any potentially healthy tissue surrounding a tumor,

The use of detection techniques having increased sensitivity, such as Remain spectroscopy, may be appropriate for the interrogation of tissue in order to evaluate a status of the tissue (e.g. healthy vs. unhealthy). However, existing Raman spectroscopy systems are not readily compatible for use intraoperatively, and have in the past required a tissue sample to be first obtained, for example via biopsy, for subsequent remote (i.e. outside of the operating room) testing. Additionally, the steps of measuring Raman spectra with acceptable signal-to-noise ratio and characterizing each of the Raman spectra measured, have to date been too time-consuming, thereby further rendering the use of Raman spectroscopy riot well suited for use during a live surgery.

### SUMMARY OF THE INVENTION

The aforementioned problems associated with the prior art, including but not limited to, the inability to accurately, repeatably and quickly perform intraoperative analysis of biological tissue for the purposes of determining a cancer status of the tissue in real-time are addressed by the solutions provided by the systems and methods of the present invention described herein.

There are therefore hereby provided methods and systems for assessing a cancer status of biological tissue in real-time, in order to be practical for in vivo applications such as live surgery (i.e. intraoperatively).

As will be seen, although the system and methods of the present disclosure may be useful in other pathologies, the present system has been found to be particularly useful in the intraoperative detection, in real-time, of brain tumors such as glioblastomas.

The methods and systems provided herein involve the use of a fiber-optic Raman spectroscopy system which generates, intraoperatively and in real-time, a Raman spectrum which is indicative of a Raman spectroscopy response of the biological tissue upon interrogation with a hand-held fiber-optic probe of the fiber-optic Raman spectroscopy system. Due to its portability, the hand-held fiber-optic probe is manipulable bye surgeon in order to interrogate biological tissue of a patient in situ and intraoperatiyely.

Concurrently with the use of the fiber-optic Raman spectroscopy system, the methods and systems disclosed herein can assess the cancer status of the biological tissue by making a comparison of the Raman spectrum to reference data using a boosted tree classification algorithm. The boosted tree classification algorithm is a classification algorithm which determines classification criteria independently from a user's input, based on the reference data, with which the Raman spectrum is to be compared in order to determine the cancer status of the interrogated biological tissue. The reference data are previously determined based on a set of reference Raman spectra indicating Raman spectroscopy responses of reference biological tissues wherein each of the reference biological tissues is associated with a known cancer status typically determined with conventional methods.

The methods and systems can thus benefit from the portability of the fiber-optic probe, The accuracy of Raman spectroscopy, and the efficiency of the boosted tree classification algorithm in order to provide methods and systems which are useable intraoperatively and in real time in order to permit the in vivo detection of unhealthy tissue that would not be readily identified intraoperative using conventional methods.

In an aspect of the present disclosure, there is provided a method for assessing a cancer Status of biological tissue, the method comprising the steps of: obtaining a Raman spectrum indicating a Raman spectroscopy response of the biological tissue, the Raman spectrum captured using a fiber-optic probe of a fiber-optic Raman spectroscopy system; inputting the Raman spectrum into a boosted tree classification algorithm of a computer program, and using the boosted tree algorithm for comparing, in real-time, the captured Raman spectrum to reference data and assessing the cancer status of the biological tissue based on said comparison, the reference data being previously determined based on a set of reference Raman spectra indicating Raman spectroscopy responses of reference biological tissues wherein each of the reference biological tissues is associated with a known cancer status; and genarciing a real-time output indicating the assessed cancer status of the biological tissue.

Further in accordance with this aspect, the method is conducted intraoperativety, and the step of obtaining the Raman spectrum includes intraoperatively obtaining the Raman spectrum from the biological tissue in vivo, and the step of generating includes intraoperatively generating the real-time output.

Further in accordance with this aspect, the step of using the boosted tree classification algorithm further comprises determining classification criteria for each one of a plurality of decision trees of the boosted tree classification algorithm based on the reference data,

Still further accordance with one or more of these aspects, the step of using the boosted tree classification algorithm further comprises determining an optimal number of decision trees.

Still further in accordance with this aspect, the optimal number of decision trees is eight.

Still further in accordance with one or more of these aspects, the reference data are previously determined in a training process of the boosted tree algorithm using the set of reference Raman spectra.

Still further in accordance with one or more of these aspects, the method further comprises obtaining two or more Raman spectra for the biological tissue, averaging the two or more Raman spectra to produce an averaged Raman spectra representative of the biological tissue, and providing the averaged Raman spectra to the boosted tree classification algorithm for comparing the averaged Raman spectra to the reference data.

Still further in accordance with one or more of these aspects, the method further comprises obtaining at least one additional signal characteristic representative of the biological tissue and inputting said at least one additional signal characteristic into the boosted tree classification algorithm, said at least one additional signal characteristic including diffused reflectance spectroscopy and fluorescence spectroscopy.

Still further in accordance with this aspect, the method further comprises using the fiber-optic probe to capture said at least one additional signal characteristic.

Still further in accordance with one or more of these aspects, the method further comprises using a computer-assisted surgery system in communication with the fiber-optic Raman spectroscopy system to determine at least one of position and orientation of the fiber-optic probe in a three dimensional surgical field.

Still further in accordance with this aspect, the method further comprises using the computer-assisted surgery system to determine a three dimensional spatial position of the biological tissue at the moment of each interrogated using the fiber-optic probe.

Still further in accordance with one or more of these aspects, the biological tissue is brain tissue, and the method includes intraoperatively assessing the cancer status of the brain tissue during neurosurgery,

in another aspect, there is provided a system for assessing a cancer status of biological tissue, the system comprising: a fiber-optic Raman spectroscopy system including a fiber-optic probe, the fiber-optic Raman spectroscopy system generating at least a portion of at least one Raman spectrum after interrogating the biological tissue in real-time with the fiber-optical probe, the a least one Raman spectrum indicating a Raman spectroscopy response of the biological tissue; and a computer comprising a processor coupled with a cornputer-readaktile memory, the computer-readable memory being configured for storing the at least one Raman spectrum and computer executable instructions that, where executed by the processor, perform the steps of: using a boosted tree algorithm for intraoperatively comparing, in real-time, the at least one Raman spectrum to reference data and assessing the cancer status of the biological tissue based on said comparison, the reference data being previously determined based on a set of reference Raman sprectra indicating Raman spectroscopy responses of reference biological tissues wherein each of the reference biological tissues is associated with a known cancel status; and intraoperatively generating a real-time output indicating the cancer status of the biological tissue.

Further in accordance with this aspect, the system is used intraoperatively, and the step of generating the real-time output performed by the computer executable instructions includes intraoperatively generating the real-time output, the real-time output including at least one of a visual and audible signal indicative of the cancer status of the biological tissue.

Further in accordance with this aspect, the fiber-optic probe is hand-held.

Further in accordance with this aspect, the computer executable instructions further comprises a step of determining classification criteria for each one of a plurality of decision tree of the boosted tree classification algorithm based on the reference data.

Still further in accordance with this aspect, the computer executable instructions further comprises a step of determining an optimal number of decision trees.

Still further in accordance with this aspect, the optimal number of decision trees is eight.

Still further in accordance with one or more of these aspects, the reference data are previously determined in a training process of the boosted tree algorithm using the set of reference Raman spectra.

Still further in accordance with one or more of these aspects, the computer executable instructions further comprises steps of averaging the at least one Raman spectrum generated by the fiber-optic Raman spectroscopy system to produce an averaged Raman spectra representative of the biological tissue, and providing the averaged Raman spectra to the boosted tree classification algorithm for comparing the averaged Raman spectra to the reference data.

Still further in accordance with one or more of these aspects, the system further comprises at least one of a fiber-optic diffused reflectance spectroscopy system and a fiber-optic fluorescence spectroscopy system, wherein the diffused reflectance spectroscopy system generates at least one diffused reflectance spectrum indicative of a diffused reflectance spectroscopy response of the biological tissue and the fluorescence spectroscopy system generates at least one fluorescence spectrum indicative of a fluorescence spectroscopy response of the biological tissue.

Still further in accordance with one or more of these aspects, the computer executable instructions further comprise a step of using said at least one additional signal characteristic into the boosted tree classification algorithm, said at least one additional signal characteristic including at least one of the diffused reflectance spectroscopy spectrum and fluorescence spectroscopy spectrum.

Still further in accordance with this aspect, the fiber-optic probe is configured to capture at least one of the diffused reflectance spectroscopy response and the fluorescence spectroscopy response.

Still further in accordance with one or more of these aspects, the system further comprises a computer-assisted surgery system in communication with the fiber-optic Raman spectroscopy system to determine at least one of position and orientation of the fiber-optic probe in a three dimensional surgical field.

Still further in accordance with this aspect, using the computer-assisted surgery system to determine a three dimensional spatial position of the biological tissue at the moment of each interrogated using the fiber-optic probe.

Still further in accordance with one or more of these aspects, the biological tissue is brain tissue, and the system is operable to intraoperatively assess the cancer status of the brain tissue during neurosurgeiy in another aspect, there is provided a method for intraoperatively assessing a cancer status of biological tissue, the method comprising the steps of: positioning a hand-held fiber-optic probe of a fiber-optic Raman spectroscopy system proximate to the biological tissue to be assessed; interrogating the brain tissue in real-time using the fiber-optic probe of the fiber-optic Raman spectroscopy system to produce at least a portion of a Raman spectrum indicating a Raman spectroscopy response of the biological tissue; using a boosted tree classification algorithm for comparing the Raman spectrum to reference data and assessing the cancer status of the biological tissue based on said comparison, the reference data being previously determined based on a set of reference Raman spectra indicating Raman spectroscopy responses of reference biological tissues wherein each of the reference biological tissues is associated with a known malignancy; and intraoperatively generating a real-time output indicating the cancer status of the biological tissue.

Further in accordance with this aspect, the method further comprises reselecting the biological tissue upon determining that the cancer status of the biological tissue is indicative of malignancy.

Still further in accordance with this aspect, the step of using the boosted tree classification algorithm further comprises determining classification criteria for each one of a plurality of decision tree of the boosted tree classification algorithm based on the reference data.

Still further in accordance with this aspect, the step of using the boosted tree classification algorithm further comprises determining an optimal number of decision trees.

Still further in accordance with this aspect, the optimal number of decision trees is eight.

Still further in accordance with one or more of these aspects, the reference data are previously determined in a training process of the boosted tree algorithm using the set of reference Raman spectra.

Still further in accordance with one or more of these aspects, the method further comprises obtaining two or more Raman spectra for the biological tissue, averaging the two or more Raman spectra to produce an averaged Raman spectra representative of the biological tissue, and providing the averaged Raman spectra to the boosted tree classification algorithm for comparing the averaged Raman spectra to the reference data.

Still further in accordance with one or more of these aspects, the method further comprises obtaining at least one additional signal characteristic representative of the biological tissue and using said at least one additional signal characteristic into the boosted tree classification algorithm, said at least one additional signal characteristic including diffused reflectance spectroscopy and fluorescence spectroscopy.

Still further in accordance with this aspect, the method further comprises using the fiber-optic probe to capture said at least one additional signal characteristic.

Still further in accordance with one or more of these aspects, the method further comprises using a computer-assisted surgery system in communication with the fiber-optic Raman spectroscopy system to determine at least one of position and orientation of the fiber-optic probe in a three dimensional surgical field.

Still further in accordance with this aspect, the method further comprises using the computer-assisted surgery system to determine a three dimensional spatial position of the biological tissue at the moment of each interrogated using the fiber-optic probe.

Still further in accordance with one or more of these aspects, the biological tissue is brain tissue, and the method includes intraoperatively assessing the cancer status of the brain tissue during neurosurgery.

In another aspect, there is disclosed a computer program comprising program code for use in a computer, the computer program causing the computer, when executed on the computer, to: obtain at least one Raman spectrum indicating a Raman spectroscopy response of biological tissue captured with a fiber-optic probe of a fiber-optic Raman spectroscopy system; use a boosted tree algorithm to compare the at least one Raman spectrum to reference data and assessing the cancer status of the biological tissue based on said comparison, the reference data being previously determined based on a set of reference Raman spectra indicating Raman spectroscopy responses of reference biological tissues wherein each of the reference biological tissues is associated with a known cancer status; and intraaperatively generate a real-time output indicating the cancer status of the biological tissue.

Further in accordance with this aspect, the program code further causes the computer to determine classification criteria for each one of a plurality of decision tree of the boosted tree classification algorithm based on the reference data.

Still further in accordance with this aspect, the program code further causes the computer to determine an optimal number of decision trees.

Still further in accordance with this aspect, the optimal number of decision trees is eight.

Still further in accordance with one or more of these aspects, the reference data are previously determined in a training process of the boosted tree algorithm using the set of reference Raman spectra.

Still further in accordance with one or more of these aspects, the program code further causes the computer to average the at least one Raman spectrum captured with the fiber-optic Raman spectroscopy system to produce an averaged Raman spectra representative of the biological tissue, and to provide the averaged Raman spectra to the boosted tree classification algorithm for comparing the averaged Raman spectra to the reference data.

Still further in accordance with one or more of these aspects, the program code further causes the computer to obtain at least one additional signal characteristic representative of the biological tissue and to use said at least one additional signal characteristic into the boosted tree classification algorithm, said at least one additional signal characteristic including diffused reflectance spectroscopy and fluorescence spectroscopy,

Still further in accordance with one or more of these aspects, the program code further causes the computer to determine at least one of position and orientation of the fiber-optic probe in a three dimensional surgical field using tracking data of associated with a computer-assisted surgery system in communication with the fiber-optic Raman spectroscopy system.

Still further in accordance with this aspect, the program code further causes the computer to determine a three dimensional spatial position of the biological tissue at the moment of each interrogated using the fiber-optic probe,

In another aspect, there is provided a computer program product for assessing a cancer status of biological tissue, the computer software product comprising: a computer-readable memory configured for storing at least one Raman spectrum indicating a Raman spectroscopy response of the biological tissue interrogated in vivo using a fiber-optic probe of a fiber-optic Raman spectroscopy system and computer executable instructions that when executed by a processor perform the steps of: using a boosted tree algorithm for comparing the at least one Raman spectrum to reference data and assessing the cancer status of the biological tissue based on said comparison, the reference data being determined based on a set of reference Raman spectra indicating Raman spectroscopy responses of reference biological tissues wherein each of the reference biological tissues is associated with a known cancer status; and intraoperatively generating a real-time output indicating the cancer status of the biological tissue.

Further in accordance with this aspect, the computer executable instructions further cause the processor to perform the step of determining classification criteria for each one of a plurality of decision tree of the boosted tree classification algorithm based on the reference data.

Still further in accordance with this aspect, the computer executable instructions further cause the processor to perform the step of determining an optimal number of the decision trees.

Still further in accordance with this aspect, the optimal number of decision trees is eight.

Still further in accordance with one or more of these aspects, the reference data are previously determined in a training process of the boosted tree algorithm using The set of reference Raman spectra.

Still further in accordance with one or more of these aspects, the computer executable instructions further cause the processor to perform the steps of averaging the at least one Raman spectrum captured with the fiber-optic Raman spectroscopy system to produce en averaged Raman spectra representative of the biological tissue, and providing the averaged Raman spectra to the boosted tree classification algorithm for comparing the averaged Raman spectra to the reference data.

Still further in accordance with one or more of these aspects, the computer executable instructions further cause the processor to perform the step of obtaining at least one additional signet characteristic representative of the biological tissue and using said at least one additional signal characteristic into the boosted tree classification algorithm, said at least one additional signal characteristic including diffused reflectance spectroscopy and fluorescence spectroscopy,

Still further in accordance with one or more of these aspects, the computer executable instructions further cause the processor to perform the step of determining at least one of position and orientation of the fiber-optic probe in a three dimensional surgical field using tracking data of associated with a computer-assisted surgery system in communication with the fiber-optic Raman spectroscopy system,

Still further in accordance with this aspect, the computer executable instructions further cause the processor to perform the step of determining a three dimensional spatia; position of the biological tissue at the moment of each interrogated using the fiber-optic probe.

In yet each other aspect, there is provided a computer implemented method for assessing a cancer status of biological tissue, comprising the steps of: obtaining at least one Raman spectrum indicating a Raman spectroscopy response of the biological tissue interrogated in vivo using a fiber-optic probe of a fiber-optic Raman spectroscopy system; using a boosted tree classification algorithm for comparing the at least one Raman: spectrum to reference data and assessing, in real-time, the cancer status of the biological tissue based on said comparison, the reference data being previously determined on a set of reference Raman spectra indicating Raman spectroscopy responses of reference biological tissues wherein each of the reference biological tissues is associated with a known cancer status; and intraoperatively generating a real-time output indicating the assessed cancer status of the biological tissue.

Further in accordance with this aspect, the step of using the boosted tree classification algorithm further comprises determining classification criteria for each one of a plurality of decision tree of the boosted tree classification algorithm based on the reference data.

Still further in accordance with this aspect, the step of using the boosted tree classification algorithm further comprises determining an optimal number of the decision trees,

Still further in accordance with this aspect, the optimal number of decision trees is eight.

Still further in accordance with one or more of these aspects, the reference data are previously determined in a training process of the boosted tree algorithm using the set of reference Raman spectra,

Still further in accordance with one or more of these aspects, the method further comprises obtaining two or more Raman spectra for the biological tissue, averaging the two or more Raman spectra to produce an averaged Raman spectra representative of the biological tissue, arid providing the averaged Raman spectra to the boosted tree classification algorithm for comparing the averaged Raman spectra to the reference data.

Still further in accordance with one or more of these aspects, the method further comprises obtaining at least one additional signal characteristic representative of the biological tissue and using said at least one signal characteristic into the boosted tree classification algorithm, said as least one additional signal characteristic including diffused reflectance spectroscopy and fluorescence spectroscopy.

Still further in accordance with this aspect, the method further comprises using the fiber-optic probe to capture said at least one additional signal characteristic.

Still further in accordance with one or more of these aspects, the method further comprises using a computer-assisted surgery system in communication with the fiber-optic Raman spectroscopy system to determine at least one of position and orientation of the fiber-optic probe in a three dimensional surgical field.

Still further in accordance with this aspect, the method further comprises using the computer-assisted surgery system to determine a three-dimensional spatial position of the biological tissue at the moment of each interrogated using the fiber-optic probe.

Still further in accordance with one or more of these aspects, the biological tissue is brain tissue and the method includes intraoperatively assessing the cancer status of the brain tissue during neurosurgery.

### BRIEF DESCRIPTION OF THE DRAWINGS

Reference is now made to the accompanying figures in which:

FIG. 1 is a schematic view of a system for assessing a cancer status of biological tissue, in accordance with an embodiment;

FIG. 2 is a flowchart of a method for assessing a cancer status of biological tissue, in accordance with an embodiment;

FIG. 3 is a schematic view of an example of a fiber-optic Raman spectroscopy system, in accordance with an embodiment;

FIG. 4 is a schematic and exploded perspective view of a fiber-optic probe used with the fiber-optic Raman spectroscopy system of FIG. 3, in accordance with a particular embodiment:

FIG. 5 is a perspective view of the fiber-optic probe of the present fiber-optic Raman spectroscopy system in use intraoperatively;

FIG. 6A is a magnetic resonance image (MRI) of a side view of a brain having a tumor;

FIG. 6B snows MRIs, pathology images and Raman spectra associated with three probe inspection sites of the tumor shown in FIG. 6A;

FIG. 6C shows MRIs, pathology images and Raman spectra associated with three other probe inspection sites of the tumor shown in FIG. 6A;

FIG. 6D shows a MRI of a top view of a brain tumor and associated pathology images;

FIG. 6E shows images of a fiber-optic probe interrogating brain tissue grade 2 tumor and a grade 4 tumor;

FIG. 7 is a graph showing a Raman spectrum for different cancer statuses, in accordance with an embodiment;

FIG. 8 is a graph showing a receiver operating characteristic of a system far assessing a cancer status of a biological tissue, in accordance with an embodiment

FIG. 9 is a three-dimensional graph showing a principal components analysis, in accordance with an embodiment;

FIG. 10 is a schematic view of a system for assessing a cancer status of a biological tissue, in accordance with an embodiment:

FIG. 11 is a schematic and exploded view of an example of a fiber-optic probe, in accordance with an embodiment; and

FIG. 12 is a graph showing Raman spectra associated with Raman spectroscopy responses of different molecules.

### DETAILED DESCRPTION

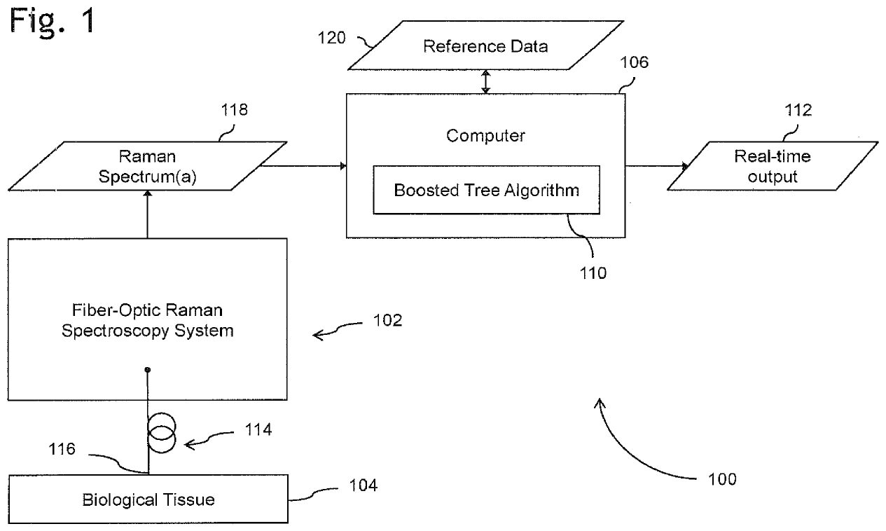

Now referring to the drawings and in particular to FIG. 1, the present system for intraoperatively assessing, in real-time, a cancer status of biological tissue is shown generally at 100.

Broadly described, the system 100 has a fiber-optic Raman spectroscopy system 102 for interrogating biological tissue 104 in vivo and a computer 106, coupled to the fiber-optic Raman spectroscopy system 102 via a suitable interface (e.g. LabVIEW™), for assessing the cancer status of the biological tissue 104, intraoperatively and in real time. As will be seen in further detail below, the computer system 106 is programmed with software which uses a boosted tree classification algorithm 110 for the purposes of assessing the cancer status of the tissue interrogated using the fiber-optic Raman spectroscopy system 102. Once the cancer status is assessed, the computer 106 generates a real-time output 112 indicating the assessed cancer status, which can then be used for guiding a surgeon during a live surgery, for instance.

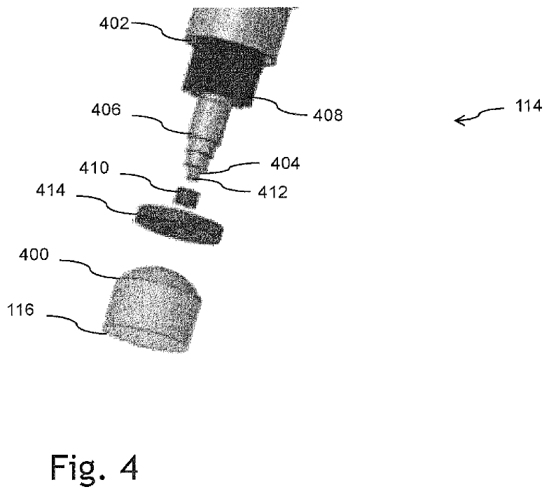

Still referring to FIG. 1, the fiber-optic Raman spectroscopy system 102 has a band-held fiber-optic probe 114, which is designed to be manipulable in viva. The fiber-optic probe 114 is handheld and has a small footprint, such that it can be readily manipulated by a surgeon or other operator with a single hand. The fiber-optic probe 114 includes an interrogation tip 116 (best shown in FIG. 4) which is to be positioned on or proximate to the biological tissue 104 to be interrogated. Once the fiber-optic probe 114 is so positioned, the fiber-optic Raman spectroscopy system 102 is actuated by a user (either the surgeon his/her self or another operator) to perform a Raman spectroscopy interrogation and generate a Raman spectrum (or Raman spectra) 118 accordingly. This is achieved, for example, by using the fiber-optic probe 114 to direct monochromatic laser light (typically in the near infrared spectrum) onto the tissue and to collect the resulting light spectrum given off by the tissue following inelastic scattering interaction of the photons of the incident laser light with the molecular content of the cells of the tissue. The Raman spectrum 118 so produced indicates a Raman spectroscopy response of the interrogated biological tissue 104. The Raman spectrum 118 generated by the fiber-optic Raman spectroscopy system 102 is then transmitted to the computer 106 where it is compared to reference data 120 using software which is based on a boosted tree classification algorithm 110, as will be discussed further below, in order to assess the cancer status of the biological tissue 104 via the captured Raman spectrum 118. The boosted tree classification algorithm 120 can rely on separate band(s) of the Raman spectrum to assess the cancer status of the biological tissue 104. However, in at least one embodiment the boosted tree classification algorithm 120 relies on a totality of the Raman spectrum 118 measured in order to factor an appropriate amount of molecular contributions.

The reference data 120 comprises a previously determined set of reference Raman spectra indicating Raman spectroscopy responses of reference biological tissues, wherein each of the reference biological tissues is associated with a known cancer status using blinded neuropathological analysis of each biopsy made on the reference biological tissues. In an embodiment, the reference data 120 is obtained by conducting a training process of the boosted tree algorithm 110 on the set of reference Raman spectra. For instance, in an exemplary set of reference Raman spectra, reference biological tissue #1 to #10 might be associated with grade 2 cancerous tissues while reference biological tissue #11 to 20 might be associated with healthy tissues. It is contemplated that reference Raman spectra captured are calibrated relative to the fiber-optic probe 114 used for capturing the reference Raman spectra. Accordingly, reference Raman spectra captured with different fiber-optic probes 114, and using multiple systems 100 in use in different locations, can be compared and used in the methods described herein.

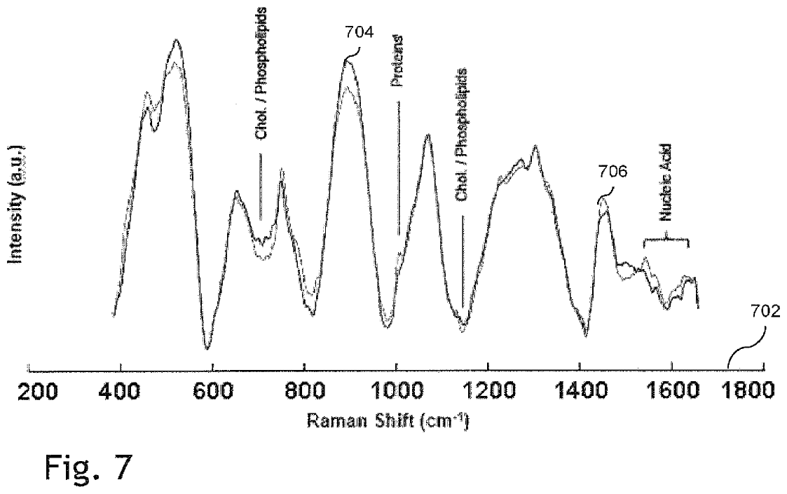

While a variety of classification algorithms have been used to analyze Raman spectra in the past, the boosted tree classification algorithm 110 was specifically found to provide advantageous overall performance. Indeed, the boosted tree classification algorithm is not limited to analysis of specific bands of the Raman spectrum 118 but also permits an analysis which employs the full Raman spectrum 118 in its entirety. It was found that the boosted tree classification algorithm 110 has en increased robustness towards noise in reference Raman spectra as well as in the captured Raman spectra 118. The robustness to noise is particularly useful given the rarity of the Raman spectroscopy response relative to the background signal. The boosted tree classification algorithm 110 does not make assumptions about feature independence and performs consistently even with a large amount of spectral information along a Raman shift axis 702 (shown in FIG. 7). The boosted tree classification algorithm 110 operates by constructing an ensemble of decision trees from the reference data 120, also sometimes referred to as “training data”. Each decision tree has classification criteria and operates on the residual of the classification determined by a previous decision tree. Using the reference data, the boosted tree classification algorithm 110 determines the classification criteria using a leave-one-out cross-validation approach, for instance. Cross-validation analysis was also used to determine the optimal number of decision trees for use with the boosted tree classification algorithm 110. Although any suitable number of trees may be used, using eight decision trees was found particularly appropriate. Selecting the number of decision trees used in the boosted tree classification algorithm 110 helps to reduce over-fitting the reference data while maintaining a complexity sufficient to suitably assess the cancer status associated with a Raman spectrum. In one embodiment, the classification criteria of the boosted tree classification algorithm are a weighted sum of comparisons at different points along the generated Raman spectra 118.

This, robustness to noise made possible by the use of the boosted tree classification algorithm can allow for a Raman spectroscopy response to be acquired during a relatively short period of time. Indeed, it is rioted that the present system allows for acquisition times in the order of 0.05-0.02 seconds, which can be sufficient for generating Raman spectra 118 with a sufficient signal-to-noise ratio. In one embodiment, it is also noted that comparing the Raman spectra 118 using the boosted tree classification algorithm 110 can be performed within one second. Consequently, given the short period of time between the time at which the Raman spectra 118 is acquired with the fiber-optic Raman spectroscopy system 102 and the time at which an output 112 indicative of the cancer status is generated by the computer 100, the system 100 is capable of performing in real-time, which can be practical for guiding and/or assisting a surgeon intraoperatively. Indeed, the present system 100 requiring such a short period of time to provide the real-time output 112 helps to reduce undesirable delays during a live surgery, thereby making its use intraoperatively, in real-time, possible.

The boosted tree classification algorithm 110 can be programmed using known programming languages such as MATLAB™, C++ or any language found suitable for treating data. When programmed in MATLAB™, the “RobustBoost” option of the “fit ensemble” function can be used for determining the reference data 120 with the set of reference Raman spectra, while the “predict” function can be used in order to assess the cancer status of the biological tissue 104 using the generated Raman spectrum 118 based on the predetermined reference data 120.

More specifically, the “fitensemble” can use inputs such as a training data matrix comprising the set of reference Raman spectra wherein each row is a reference Raman spectra. The “fitensemble” can also have an input such as a training classes vector embodied as a column vector of tissue classes or statuses wherein each row is either 1 or 0, for instance, indicating that the reference Raman spectrum of the corresponding row in the training data matrix is associated with cancer tissue or normal tissue, respectively. The “fitensemble” can also have an input such as a classification method wherein “RobustBoost” is chosen so as to force use of a boosted tree classification algorithm. The “fitensemble” can also have an input such as a number of learners which corresponds to the number of trees used, since trees are used as learners in the boosted tree classification algorithm. Accordingly, the “fitensemble” can have a type of learner which is set to “tree” in order to use the boosted tree classification algorithm. This implementation of boosted trees classification algorithm operates by constructing an ensemble of decision trees based on the training data with known classes. The classification can then be fed reference data (i.e. reference Raman spectra), where the classes are unknown, and will predict the classes of the reference data. By using ‘Robustboost’, measurements with large negative margins are given less weight than other measurements, This minimizes the negative effect that mislabeled training data may have on classification accuracy. This can be relevant for the application of cancer detection, since variance in pathology analysis or tissue sampling have the potential to give training data.

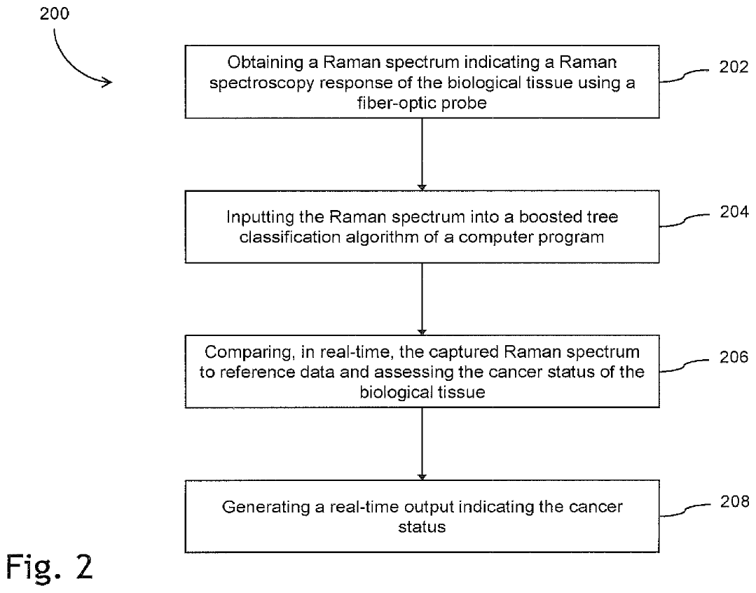

FIG. 2 shows a flowchart of an exemplary method 200 for assessing the cancer status of the biological tissue, in accordance with an embodiment of the present disclosure. The method 200 generally includes obtaining a Raman spectrum indicating a Raman spectroscopy response of the biological tissue using a fiber-optic probe at 202. It is understood that in order to generate an acceptable Raman spectrum 118, the interrogation tip 116 of the fiber-optic probe 114 is positioned in dose proximity to, or in contact with, the biological tissue 104, where it is maintained in a substantially stationary position during acquisition of the Raman spectrum 118. The method 200 also includes the step 204 of inputting the Raman spectrum into the boosted tree classification algorithm 210 of a computer program. The method 200 also includes the step of comparing, in real-time, the captured Raman spectrum to reference data and assessing the cancer status of the biological tissue at 206. After said comparison, the method 200 includes the step of generating a real-time output indicating the cancer status cite biological tissue at 208.

In an embodiment, the biological tissue 104 is brain tissue wherein the fiber-optic probe 114 is inserted through the skull of a patient in order to identify the cancer status of a multitude of regions within the patient's brain. When the fiber-optic probe 114 is positioned proximate to brain tissue 104 that the surgeon desires to interrogate, the fiber-optic Raman spectroscopy system 102 is actuated to generate a Raman spectrum 113 which is indicative of a Raman spectroscopy response of the interrogated brain tissue 104. In a relatively short period of time, less than 1 second for example, the system 100 can process the Raman spectrum 118 in order to determine the cancer status of the interrogated brain tissue 104 using a software program comprising the boosted tree classification algorithm 110 and then generate the real-time output 112 indicating the cancer status of the interrogated brain tissue 104. The real-time output 112 produced by the system 100 can then guide the surgeon as to whether he/she should leave the biological tissue 104 within the brain if it is assessed to be healthy, or remove the biological tissue 104 from the brain if it is assessed to be unhealthy, for instance. This process can be repeated as required, for example in order to determine precise margins of a glioblastoma tumor, for example.

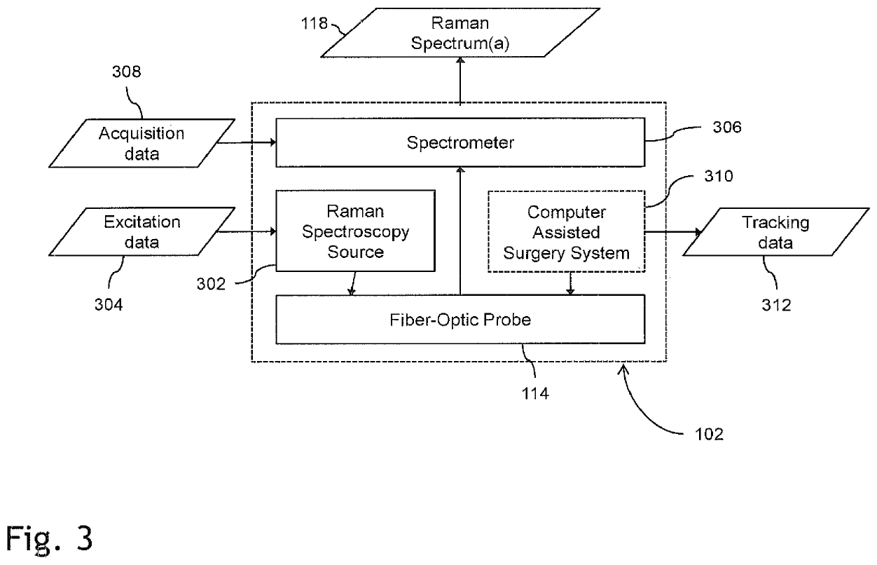

Referring now to FIG. 3. an example of the fiber-optic Raman spectroscopy system 102 is depicted, although it is understood that other appropriate types of fiber-optic Raman spectroscopy system 102 may be provided. In the embodiment shown, the fiber-optic Raman spectroscopy system 102 has a Raman spectroscopy source 302 transmitting Raman excitation light to the biological tissue 104 via the fiber-optic probe 114. The Raman spectroscopy source 302 can be provided in the Porro of a near-infrared (NIR) and more particularly in the form of a laser diode emitting at 785 nm or a solid-state Nd:YAG laser source emitting at 1064 nm, for instance. In the embodiment shown, the emission of the Raman excitation light is controlled via excitation data 304 provided by the computer 106 of the system 100. The excitation data 304 may comprise instructions concerning laser power and the like.

Still referring to the embodiment shown in FIG. 3, the fiber-optic Raman spectroscopy system 102 has a spectrometer 306 generating the Raman spectrum 118 after reception of the Raman spectroscopy response caused by the propagation of the Raman excitation light in the biological tissue 104. The spectrometer 306 can be provided in the form of a charge-coupled device (CCD) spectroscopic detector. One exemplary manufacturer of such a spectrometer 306 is Andor Technology™. In an embodiment, it is useful to cool down the CCD of the spectrometer 306 to −40° Celsius in an attempt to reduce noise in the measurements. As depicted, the spectrometer 306 is controlled via acquisition data 308 provided by the computer 103 of the system 100. The acquisition data 308 may comprise instructions concerning the acquisition time of each Raman spectroscopy interrogation as well as the temperature to which the CCD is to be cooled down, for instance.

As shown in FIG. 3, the fiber-optic Raman spectroscopy system 102 has an optional computer-assisted surgery (CAS) system 310 which is used to track a spatial coordinates of the fiber-optic probe 114 during the measurements.

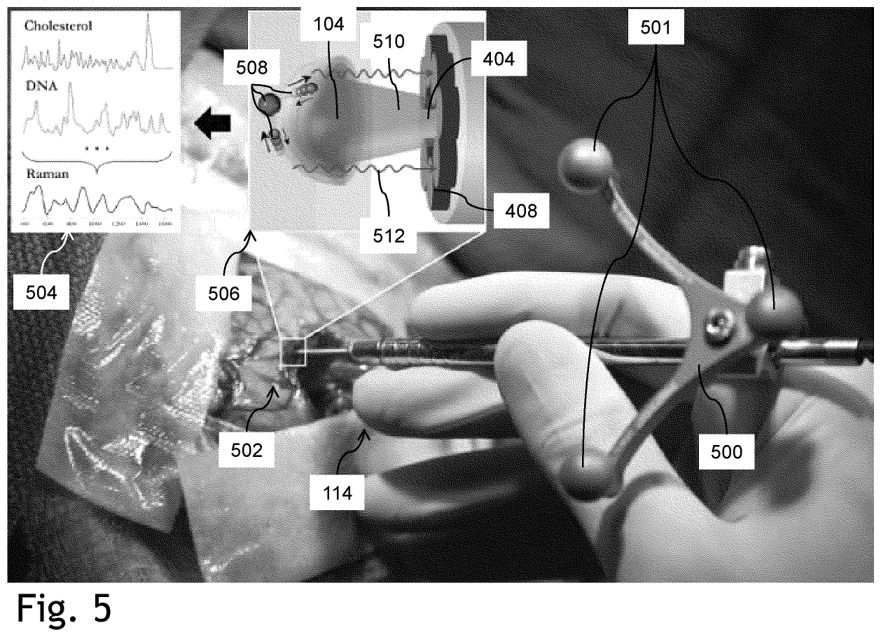

Referring concurrently to FIG. 3 and to FIG. 5, the CAS system 310 is capable of real time location and tracking of at least one trackable member 500 in a surgical field, each having a distinctive set of identifiable markers 501 thereon. These trackable members 500 are thus affixed both to the surgical tools, such as the fiber-optic probe 114, employed within the surgical field and in operable communication with the GAS system 310, for instance. At least one reference trackable member, including similar markers 501, may also be affixed to the surrounding bone (such as the skull) or to a fixed reference surface (e.g, the operating table).

The CAS system 310 may generally include a computer (either distinct from the computer 106 of the system 100 or integrated therewith), a display device (not shown) in communication with the computer, and a tracking system (not shown) also in communication with the computer. The tracking system may be an optical tracking system, using infra-red cameras to identify markers 501 for example, however any other type of tracking system can also be used, such as ones which employ wireless inertial-based sensors, laser, ultrasound, electromagnetic or RF waves for example, to locate the position of the identifiable markers 501 of the tracking members 500 within range of the sensing devices of the CAS system 310, and therefore permit the identification of at least one of the position and orientation to the instrument (e.g, the hand-held fiber-optic probe 114) to which the markers 501 are affixed.

The CAS system 310 is capable of depicting a fixed reference, a movable patient reference, and/or the fiber-optic probe 114 and any other surgical tools which may be required, on the display device (which may include a monitor for example) relative to the patient anatomy, including the bones and/or soft tissue which are also tracked in real time by the system.

The fiber-optic probe 114 may therefore optionally include at least one trackable member 500 thereon which is in communication with the cameras or position detectors of the CAS system 310. The trackable member 500 includes three retro-reflective identifiable markers 501 thereon such that the trackable member 500 is locatable and trackable by the tracking system of the CAS system 310. The CAS system 310 is thus able to determine the position, orientation and movement of the tracking member 500 (and therefore also the probe to which a bone or a skull reference is fastened) in three dimensional space and in real time. The retro-reflective identifiable markers 501 of the trackable member 500 can be removably engaged to the tracking member 500 of the fiber-optic probe 114.

The CAS system 310 typically has the optical tracking system which holds NIR cameras in a direction of the CAS trackable member 500 of the fiber-optic probe 114, which is positioned stationary relative to the fiber-optic probe 114. More specifically, the CAS system 310 may therefore be used to monitor the exact position of the interrogation tip 116 of the fiber-optic probe 114 for each Raman spectroscopy interrogation. Accordingly, each Raman spectrum generated by the spectrometer 306 can be associated with corresponding spatial coordinates so that healthy and/or unhealthy biological tissue can be located and stored. In the embodiment shown, CAS tracking data 312 is continuously forwarded to the computer 106 of the system 100 so that when the spectrometer 306 interrogates the biological tissue 104, the computer can determine and record the spatial coordinates of the fiber-optic probe 114. An example of the 3D GAS system 310 is Medtronic™'s StealthStation™ which involves the use of identifiable markers 501 (as shown in FIG. 4) disposed on the fiber-optic probe 114 and the use of an optical CAS apparatus which compotes the spatial coordinates of the identifiable markers 501 in real-time. Other suitable CAS systems 310 may be deemed appropriate depending on the circumstances.

It is noted that the tracking data 312 can be used to extract and store the three-dimensional) spatial positions of the probe interrogation site or sites. The tracking data 312 can be used subsequent to the live surgery, for instance, for identifying a position and/or orientation of each probe interrogation site relative to the MRI. The tracking data 312 is configured so that the recorded probe interrogation sites can then be co-registered to other pre- and post-operating imaging of multiple modalities (i.e. T1 MRI, T2 MRI, DWI, DTI) to allow further comparison of the captured Raman spectra to radiological signal of the biological tissue assessed during use.

FIG. 4 shows an exploded view of an example of the fiber-optic probe 114, although it is understood that other appropriate types of fiber-optic probes 114 may be provided. As illustrated, the fiber-optic probe 114 has an outer protective cladding 402 which has enclosed therein en excitation optical-fiber 404 delivering the Raman excitation light generated by the Raman spectroscopy source 302. The fiber-optic probe 114 has an interrogation lens 400 provided at the interrogation tip 116. In this example, the excitation optical-fiber 404 is concentric relative to the outer protective cladding 402, inner guiding claddings 406 are disposed around the excitation optical-fiber 404 in order to protect the excitation optical-fiber 404. As shown, the fiber-optic probe 114 also has collection optical-fibers 408 each disposed along the excitation optical-fiber 404 and circumferentially distributed therearound. The collection optical-fibers 408 collect the Raman spectroscopy response which is caused by propagation of the Raman excitation light within the biological tissue 104. In the embodiment shown at FIG. 4, the excitation optical-fiber 404 has a band-pass (BP) filter 410 disposed at a tip 412 of the excitation optical-fiber 404 for filtering the Raman spectroscopy light in order to interrogate the biological tissue 104 at a given wavelength. The fiber-optic probe 114 also has a long-pass (LP) filter 414 concentrically surrounding the BP filter 410 in order to filter out the Raman excitation light from the light collected by the collection optical-fibers 408. In an embodiment, the BP filter 410 is narrowly centered at 785 nm when the Raman spectroscopy source 302 is the laser diode emitting at 785 nm. In this embodiment, the LP filter 414 lets pass wavelengths longer than 785 nm in order to filter out light association with the laser diode emitting at 785 nm, for instance. In view of the above, the fiber-optic probe 114 can be said to be a filtered fiber-optic probe 114. Such a filtered fiber-optic probe 114 is less influenced by background noise, ambient light and the like, which was found convenient for providing Raman spectrum 118 having acceptable signal-to-noise ratio using reduced acquisition times. An example of the filtered fiber-optic probe 114 is described in U.S. Pat. No. 8,175,423 B2 to Marple, the entire content of which is hereby incorporated by reference. Moreover, the filtered fiber-optic probe 114 can be provided by Emvision™ LLC.

In this disclosure, the term “cancer status” is understood to indicate the malignancy of the interrogated biological tissue 104. For instance, the cancer status can be either healthy or unhealthy, either cancerous or non-cancerous and it can also be indicative of the type of tumor and/or the grade of tumor thereof. Also, the real-time output 112 is understood to refer to any kind of visual indication that can inform the surgeon and/or operator of the system 100 of the malignancy of the biological tissue 104 interrogated with the fiber-optic Raman spectroscopy system 102. For instance, the real-time output 112 can be provided as a binary response wherein the real-time output 112 is positive when the biological tissue 104 is cancerous or the real-time output 112 is negative when the biological tissue healthy. In another embodiment, the real-time output 112 can be provided as a colour-coded response, for example wherein the real-time output 112 includes a red light when the biological tissue is assessed to be cancerous and a green light when the biological tissue is assessed to be healthy, wherein any shade of colour between the red and the green indicates a corresponding malignancy of the biological tissue. In another embodiment, the real-time output 112 can be provided as a numerical score, for example where the real-time output 112 is rated depending on the malignancy of the biological tissue. In another embodiment, the real-time output 112 includes an audible signal, which can be heard by the surgeon and/or operator of the system 100. This may include, for example, an audible tone which is indicative of the detection of cancerous tissue and/or that the interrogated tissue at that site is healthy. A combination of both the audible and visual outputs is also possible. Any other suitable embodiments of the real-time output 112 indicating the cancer status can be preferred depending on the circumstances.

As will be illustrated in part by examples provided below, the system 100 can be embodied using different types of fiber-optic Raman spectroscopy systems 102 and different methods of assessing the cancer status of the biological tissue 104. The system 100 can be used in different applications, and adapted to such applications via a proper selection of settings, configuration and components, for instance.

The methods and systems disclosed herein may be implemented suitably for use with a computer. For instance, the methods and systems can involve the use of a computer program comprising program code for use in a computer, wherein the computer program causes the computer to perform steps disclosed herein when the computer code is executed on the computer, Moreover, the methods and systems can involve the use of a computer program product for assessing a cancer status of biological tissue, the computer software product comprising: a computer-readable memory configured for storing at least one Raman spectrum indicating a Raman spectroscopy response of the biological tissue interrogated in vivo using a fiber-optic probe of a fiber-optic Raman spectroscopy system and computer executable instructions that when executed by a processor perform the steps disclosed herein. Further, the methods can be provided in the form of a computer implemented method for assessing a cancer status of biological tissue, comprising the steps disclosed herein, for instance.

### EXAMPLE

### Intraoperative Brain Cancer Detection using the System 100

Since the fiber-optic probe 114 can be used intraoperatively and the cancer status can be assessed in real-time, the system 100 was found convenient for use in assessing brain cancers, such as glioblastoma, wherein detecting even a low level of invasive cancer may be important.

Malignant brain tumors, particularly gliomas, derive from diverse cells of origins and are genetically heterogeneous, however they all share a distinct biological feature: aggressive diffuse invasion of tumor cells from the primary mass into the surrounding tissue. The manner in which this occurs is strikingly distinct from other high-grade solid tumors such as small-cell lung carcinoma, mammary ductal carcinoma, prostate cancer and colorectal cancers. Whereas, these more common cancers typically metastasize away from their tissue of origin through intravascular or lymphatic mechanisms, gliomas are almost never found to have metastasized away from the brain. Instead gliomas are characterized by cells which activate mechanisms more often associated with stem cells or immature neurons to actively migrate through the extracellular space of brain tissue. The highly active state of these pathways in gliomas leads to rapid invasion of diffuse cancerous cells away from the primary tumor and these cells are able to give rise to satellite tumors within the same tissue (i.e. the brain), often as far away as the other hemisphere. Thus, much more than in other cancers, the prevention of disease recurrence in brain cancers depends critically on the eradication of these invading cells, which are often very difficult to detect.

However, the present system 100 was found particularly useful during neurosurgery, because the system 100 can rapidly assess the cancer status of the brain tissue at a probe interrogation site without the need for biopsy and frozen neuropathology assessment conducted remotely from the operating room, which can disrupt conventional surgical workflows when performed several times during a surgery, for instance. Differently from other pathologies, the standard of care in brain cancer resection does not include multiple tissue biopsies around the tumor bulk to identify clean differentiation between healthy and unhealthy tissues. Therefore, although the system 100 may be useful in the other pathologies, the system 100 has been found to be particularly useful in the detection of brain tumors such as glioblastomas.

An advantage of the system 100 is to detect invasive cancer within a normal brain that may not otherwise be detectable using 5-ALA-PplX and MRI techniques. The system 100 can enable detection of invasive brain cancer in all grades of glioma, which potentially fills an important role in neurosurgical guidance.

Brain cancer cells are typically classified in World Health Organization (WHO) grades. Low-grade (WHO grade 2) gliomas are well-differentiated tumors which are characterized by acceptable-prognosis for the patient while high-grade gliomas (WHO grades 3 and 4) are undifferentiated tumors which are malignant and which carry a worse prognosis. Accordingly, the prognosis for patients with grade 2 gliomas (benign) is better than that of grade 3 and 4 gliomas because these cancers, in general, grow more slowly, have a more favorable response to adjuvant radiotherapy and chemotherapy, and most often occur in younger patients with excellent performance status who are able to tolerate the adjuvant therapies. Invariably, grade 2 cancers progress to grades 3 and 4. This understanding of the natural history of grade 2 gliomas has led to an interest in earlier and more aggressive treatments, which include surgical cytoreduction. Retrospective data suggest that maximal surgical resection provides a major survival benefit for patients with made 2 gliomas, in some cases up to additional decades. There is similarly strong evidence showing that the extent of tumor resection for grade 3 and grade 4 gliomas also affects survival. As a result, a goal of brain cancer resection is to minimize volume of residual cancer remaining after surgery to prolong survival and alleviate symptoms while minimizing the risk for neurological injury associated with the unnecessary resection of normal tissue. Attaining this goal is challenging because grade 2 to 4 gliomas are highly invasive, which is manifested by the fact that these cancers are not restricted to areas of MRI contrast uptake and/or T2 hyperintensity, for instance.

FIG. 5 shows an image of the fiber-optic probe 114 in intraoperative use for assessing the cancer status of brain tissue. The fiber-optic probe 114, provided by Emvision™ LLC, was used for single-point submillimeter Raman spectroscopy response detection in order to distinguish brain cancer tissue from healthy, normal tissue. The fiber-optic probe 114 has the excitation optical-fiber 404 and the collection optical-fibers 408 described above. The excitation optical-fiber 404 delivers light at 735 nm generated by an NIR spectrum-stabilized laser generator. The collection optical-fibers 408 of the fiber-optic probe 114 were optically coupled to a high-speed and a high-resolution spectrometer 306. The Raman spectroscopy source 302 and the spectrometer 306 were coupled to the computer system 106 to visualize generated Raman spectra in real time. The Raman spectra generated by the spectrometer 306 has a range of Raman shifts from 381 cm⁻¹ to 1653 cm⁻¹, with a spectral resolution varying between 1.6 cm⁻¹ and 2.1 cm⁻¹.

During each tumor resection procedure, the fiber-optic probe 114 was used to measure the Raman signal at several points in surgical cavity 502 as depicted in FIG. 5. The Raman (inelastic) scattering signal is several orders of magnitude smaller than that associated with Rayleigh (elastic) scattering. As a result, a challenge was to detect and isolate the tissue's inelastic scattering signal from the elastic scattering signal due to the Raman excitation wavelength of the Raman spectroscopy source at 785 nm. To do so, the filtered fiber-optic probe 114 shown in FIG. 4 was used.

The probe 114 provided a circular interrogation spot having a diameter of 0.5 mm and an area of 0.2 mm². Light transport simulations in tissue were performed using Mesh-based Monte Carlo as discussed in “Q. Fang, Mesh-based Monte Carlo method using fast ray-tracing in Plücker coordinates, Miomed. Opt. Express 1, 165-175 (2010)” and in “Q. Fang, D. R Kaeli, Accelerating mesh-based Monte Carlo method on modem CPU architecture. Biomed. Opt. Express 3, 3223-3230 (2012)”, the entire contents of which are incorporated herein, for demonstrating that an interrogation depth of the fiber-optic probe 114 associated with 95% of the Raman spectroscopy response comes from the first ˜1 mm beneath a surface of the tissue 104. The circular interrogation spot of 0.5 mm and the interrogation depth of ˜1 mm was found appropriate far resection because it is consistent with the level of precision neurosurgeons can reach using state-of-the-art neurosurgical microscopes and tissue dissection techniques.

A signal-to-noise ratio (SNR) of 15.8 was calculated for the system 100 as the ratio of the Raman peak size versus the noise, with noise defined as the difference between the maximum and minimum intensities in the baseline of the Raman spectra. The acetaminophen's (e.g., Tylenol™) Raman spectrum was used as a calibration standard for this calculation, with peaks in the spectrum chosen closest in size to those seen in the Raman spectra associated with brain tissue. This reference spectrum is used for suitably scafing the Raman shift axis of the captured Raman spectrum for proper calibration thereof.

Suitable calibration of the fiber-optic probe 114 can allow for comparing Ranan spectra measured with different fiber-optic probes 114, winch can be useful in practice. Indeed, once a given fiber-optic probe 114 is properly calibrated, the Ramar spectra captured with the given fiber-optic probe are generally exempt from artifacts associated with a response function of the given fiber-optic probe 114. Therefore, Raman spectra captured with the given fiber-optic probe can be compared to Raman spectra that would be captured with another fiber-optic probe, for instance. In an embodiment, the set of reference Raman spectra used for determining the reference data are captured using different calibrated fiber-optic probes 114.

As shown in FIG. 5, inset 504 shows the Raman spectroscopy response of different molecular species, such as cholesterol and DNA, to produce the Raman spectra of cancer versus normal brain tissue. The spectral differences occur due to the vibrational modes of various molecular species. A simple molecular vibrational mode is conceptually depicted at inset 506 where molecules 508 interact with the Raman excitation light 510 to produce a Raman spectroscopy response as shown at 512.

Before the brain cancer resection, the fiber-optic probe 114 and associated equipment were sterilized using a sterilization system such as the STERRAD™ system. The CCD of the spectrometer 306 was cooled to −40° C., and all external lighting in the operating room were turned off, with only two operating room lights (e.g. Dr. Mach™, models 380 and/or 500) were left active in the operating room. During surgery, the neurosurgeon used a white light from OPMI Pentero™ surgical microscope system sold by Zeiss™. Suitable measurement locations were selected by the neurosurgeon using MR guidance from the CAS system 310. For this experiment, a goal was to select normal brain, dense cancer, and normal brain infiltrated with invasive cancer cells at various locations in and around the tumor area detected on the MR images. Samples were acquired in both gray matter and white matter.

Before measurements using the fiber-optic probe 114, the neurosurgeon reduced blood in the area to be sampled. A measurement was then made with the fiber-optic probe 114 in direct contact with the brain tissue, with the bright-field microscope's white light turned off temporarily.

The probe interrogation sites were marked in the MRI using the CAS system 310. Given that the CAS system 310 used in this experiment involves the use of a strong NIR signal emitted by the NIR cameras, the CAS mount was temporarily pointed away from the patient while the Raman spectroscopy interrogation was performed. A reference background measurement was first taken with the Raman spectroscopy source 302 turned off with an acquisition time of 0.05 s. Then, the three Raman interrogations were performed each with an acquisition time of 0.05 s thus resulting in a total acquisition time of 0.2 s. Once transmitted to the computer system 106, the three Raman spectra are averaged with one another and the background measurement is subtracted from the averaged Raman spectrum to account for ambient light sources. The Raman spectra were then preprocessed to normalize for the laser power at which the Raman spectroscopy source was set for each captured Raman spectrum. Intrinsic tissue fluorescence was removed from the resulting Raman spectrum using a fourth-order polynomial fitting method. In an embodiment, the measured or monitored data are included in two separate text files for each patient. The first text file has raw Raman spectra and the second text file has notes concerning settings of the system 100 (e.g. acquisition time, background spectrum, averaged Raman spectrum associated with each probe interrogation site and comments of the surgeon). In another embodiment, the captured Raman spectra are processed in real-time in order to remove the background spectrum and to remove intrinsic fluorescence of the tissue (e.g, using a fourth-order polynomial) so that the Raman spectrum can be displayed to the surgeon in real-time. The real-time displayed Raman spectrum can be used for indication purposes (e.g. adjusting the laser power and/or avoiding saturation of the CCD).

Each time a Raman interrooation was made with the fiber-optic probe 114, the latter was gently placed in contact with brain tissue to ensure that no air gap existed between the interrogation tip 116 of the fiber-optic probe 114 and a surface of the biological tissue 104. This left (on white matter, or on any other brain tissue type) a temporary circular demarcation on the tissue surface, which was used by the neurosurgeon as a target location where a tissue biopsy sample was collected immediately after the Raman measurement. The sample—on average with a size of ˜0.5 mm×˜0.5 mm and a depth (from the surface) of ˜3 mm—was then removed from the patient and preserved in formalin, to be archived and analyzed by a neuropathologist at a later date.

Laser power was adjusted before each interrogation in order to account for difference in ambient light and intrinsic tissue fluorescence to avoid saturating the CCD. The laser power output as measured at the interrogation tip 116 of the fiber-optic probe 114 ranged from 37 to 64 mW. At each of the probe interrogation sites, the neurosurgeon also commented, on the basis of tissue appearance (visual assessment through a surgical microscope),navigation guidance and CAS data 312, whether the interrogated site likely corresponded to normal brain tissue (negative for cancer cells) or cancerous tissue. Those comments were recorded to allow comparison of the classification efficacy of the system 100.

On the basis of standard clinical practice, atypical cells were identified on H&E-stained sections on the basis of their morphological features, including nuclear atypia and nuclear polymorphism. As part of the standard neuropathological analysis, each tumor is also tested for the IDH1 (R132H) mutation, a known gliomarnarker. On the subset of tumors positive for the mutation, IDH1 (R132H) immunohistochemistry analyses were also conducted. Cell counting (total cell count per area, cancer cell count per area, and cancer cell burden) was done for 14 samples on the basis of H&E stain images. Further, cell counting based on immunohistochernistry was also done on n=4 invasive cancer samples from three different patients (two of the four samples belonged to the same patient) having tested positive for the IDH1 (R132H) mutation. For those samples, the normal and cancer cell (positively stained cells) count per unit area was computed, and the cancer cell burden was evaluated.

The immunohistochemistry for the IDH1 R132H antibody clone H09 (Dianova™) was performed on an automatic immunostainer BenchMark XT (Ventana™), using a pretreatment with Cell conditioning 1 (CC1) and the XT OptiView™ DAB kit. The antibody was diluted with a 1:100 ratio. Immunostains were not performed on the next serial section from the H&E; therefore, although the absolute number of cells might differ, the cancer cell burden was comparable. This example experiment was designed to reduce spatial inconsistencies between the biopsied tissue and the actual volume interrogated with Raman spectrosopy light by the system 100. The average biopsy sample surface area was the same as the surface area sampled with the probe (0.5 mm×0.5 mm). Biopsy samples were taken superficially using standard microdissection surgical instruments.

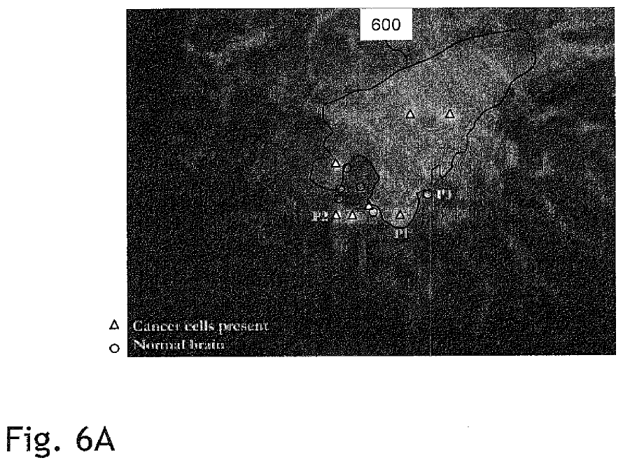

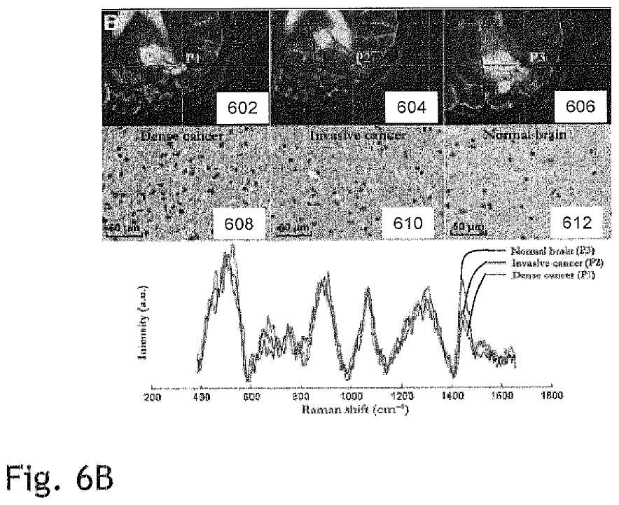

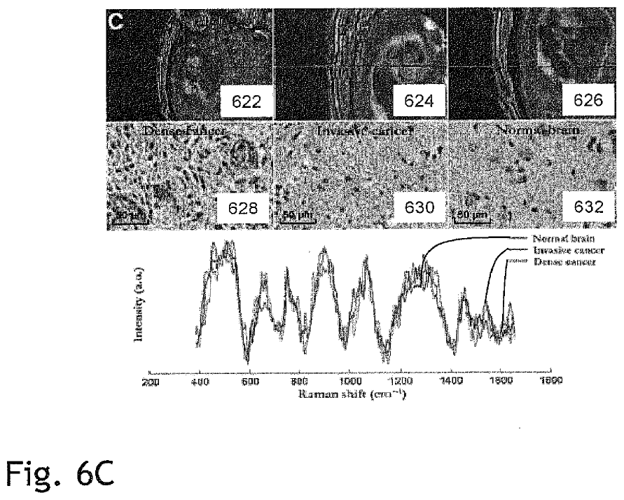

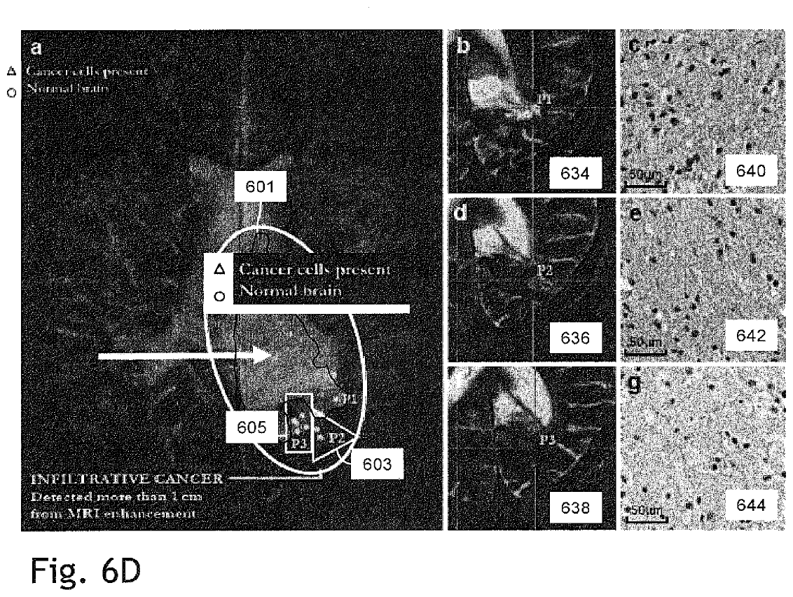

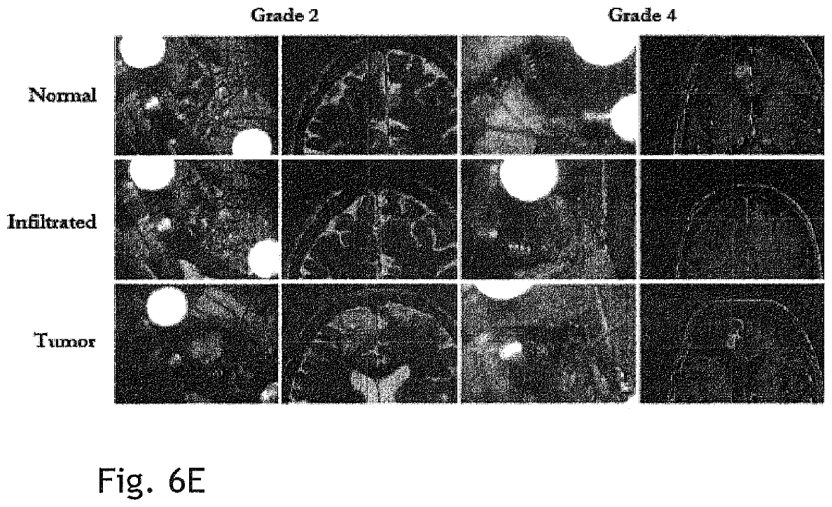

Using the system 100, a total of 161 Raman spectra were collected (see Table 1) in 17 patients with WHO grade 2 to 4 gliomas undergoing brain cancer resection. Here, emphasis was placed on interrogating brain cancer regions both within the MRI-defined dense cancer and outside (up to 1.5 cm) of the T1-gadolinium enhancing and T2-weighed hyper-intense regions in grade 2 to 4 gliomas. Although neuro-navigation techniques were used in this example experiment, MRI information was used only qualitatively for visualization purposes and for estimating the location of each Raman measurement on the preoperative images, i.e. the position of the crosshair (also referred to as ‘reticle’) shown in FIGS. 6B-D. As a result, this information, along with the inherent inaccuracies associated with the neuro-navigation CAS system 310, had an acceptable impact on correlating positions associated with biopsied samples (used for determining the reference data 120) and corresponding probe interrogation sites.

Indeed, for determining the reference data 120, each probe interrogation site was biopsied and archived for post-surgery, blinded, histopathological analysis. The surgeon was blinded to any information about the acquired Raman spectra during the resection procedure. The pathologist was blinded to any information about the Raman spectra before performing the histological analyses. Samples were excluded from analysis if they were entirely necrotic, if saturation of the CCD occurred, if they were determined by the pathologist to have substantial heterogeneity in cancer cell density (part of the sample with the presence of cancer cells and part with no cancer cells), or in the presence of noticeable signal artifacts from the CAS system 310 or room lighting. To correct far brain shift during surgery and thus increase probe backing accuracy, several landmarks using preoperative MRI before taking Raman measurements were recorded. These landmarks were then compared with a reconstructed cortical surface (from segmented preoperative MR images) and used to estimate brain shift.

In this example experiment, the blinded neuropathological analysis of each biopsy sample was performed using hematoxylin and eosin (H&E) staining. For samples arising from tumors containing the isocitrate dehydrogenase 1 (IDH1) (R132H) mutation, immunahistochernistry using an anti-IDH1 (R132H)-specific antibody was used as a complementary technique to identify cancer cells. On the basis of these neuropathological analyses, each sample was classified as either normal brain (no cancer cells present), normal brain infiltrated with invasive cancer cells (≤90% cancer cells present), or dense cancer (>90% cancer cells present (see Table 1), which was used for determining the reference data in this example experiment. For 77 of the 161 biopsy samples collected, the background could clearly be identified by The pathologist as either white matter or gray matter (n=36 samples in gray matter, n=41 samples in white matter).

Table 1 presented herebelow shows patient demographics and histological diagnoses. The diagnoses were made according to the WHO, an the basis of the consensus of pathologists and international experts, providing definition for brain tumors in cancer research, as seen in “D. N. Louis, H. Ohgaki, O. D. Wiestler, W. K. Cavenee, P. C. Burger, A. Jouvet, B. W. Scheithauer, P. Kleihues, The 2007 WHO classification of tumors of the central nervous system. Acta Neuropathol. 114, 97-109 (2007),”. For the “other” classification, only normal brain samples were used from the indicated patients; no samples with cancer cells present were acquired.

TABLE 1
Patient demographics and histological diagnoses

| | | n patients | n samples |
|---|---|---|---|
| Age (years), median (range) | 53 (30-89) | | |
| WHO grade | Grade 2 | 4 | 35 |
| | Astrocytoma | 3 | 26 |
| | Oligodendroglioma | 1 | 9 |
| | Grade 3 | 3 | 29 |
| | Astrocytoma | 1 | 10 |
| | Oligodendroglioma | 1 | 10 |
| | Oligoastrocytoma | 1 | 9 |
| | Grade 4 (GBM) | 8 | 68 |
| | Other: metastatic | 2 | 29 |
| Tissue type | Normal brain | | 66 |
| | Dense cancer | | 39 |
| | Invasive cancer cells | | 56 |
| Total | | 17 | 161 |

FIG. 6A shows a preoperative T2-weighted MRI image of a patient with a grade 2 glioma, with the probe interrogation sites identified with triangles for cancerous tissue and circles for normal tissue. Perimeter 600 delimits a grade 2 astrocytoma as identified by the preoperative MRI. It can be seen that cancer tissue can be found within the perimeter 600 but also outside the perimeter 600, which illustrates a purpose of providing the system 100. The MRI image was used only qualitatively for visualization, not for spatial registration between histology and probe interrogation sites. Sites identified by circles and triangles were interrogated with the fiber-optical probe 114 and were histologically analyzed for contributing to the reference data 120.

FIG. 6B shows 2D preoperative MRI images 602, 604 and 606 associated with probe interrogation sites P1, P2 and P3, along with corresponding pathology images 606, 610 and 612 and corresponding generated Raman spectra. The tissues interrogated at probe interrogation sites P1, P2 and P3 are associated with dense cancer, invasive cancer and normal brain, respectively.

FIG. 60 shows 2D preoperative MRI images 622, 624 and 626 associated with three probe interrogation sites in a grade 4 glioblastoma (GMB), corresponding pathology images 628, 630 and 632 and corresponding generated Raman spectra. The tissues interrogated at these three probe interrogation sites are associated with dense cancer, invasive cancer and normal brain.

FIG. 6D shows en MRI of a top view of a tumor of a brain and associated pathology images. More specifically, perimeter 601 indicates the tumor as identified with the MRI. The probe interrogation sites which correspond to cancer are enclosed by triangle 603 while the probe interrogation sites which correspond to normal brain are enclosed by rectangle 605. It can be seen in FIG. 6D that the system 100 can be used to identify cancer cells that would not have been detected with conventional techniques since these cancer cells are located well outside the perimeter 601. Indeed, FIG. 6D is a 3D volume rendering of a preoperative T2W MRI overlaid with a segmentation of the grade 2 astrocytoma delimited by the perimeter 601. Specimens P1-3 were interrogated by the system 100 and were histologically analyzed independently. Purple sample locations indicate the presence of cancer cells on the coloured view of the figure (surrounded by a triangle on the black and white view of the same figure), while green locations were negative for cancer cells on the coloured view of the figure (surrounded by a rectangle on the black and white view of the same figure). Samples for each tissue type are indicated, and corresponding pathology images are included for each.

Inset 634 of FIG. 6D shows sample location P1 within the dense cancer shown on the T2W MRI: inset 640 shows a histopathology image of dense cancer at P1; inset 636 shows sample location P2 for low density invasive cancer shown on the T2W MRI, inset 642 shows a histopathology image of low density invasive cancer at P2; inset 638 shows sample location P3 for normal brain shown on the T2W MRI; inset 644 shows a histopathology image of normal brain at P3;

FIG. 7 shows examples of Raman spectra that are generated with the system 100, in accordance with this example experiment. As illustrated, a first curve 704 shows averaged Raman spectra associated with healthy biological tissues (66 Raman spectra were averaged) while a second curve 706 shows averaged Raman specta associated with unhealthy, cancerous biological tissues (95 Raman spectra were averaged). It can be seen in FIG. 7, that differences between curves 504 and 506 are more noticeable at specific areas along the Raman shift axis 702. For instance, regions associated with cholesterol and phospholipids proximate to 700 cm⁻¹ and 1142 cm⁻¹, the breathing mode of phenylalanine in proteins near 1005 cm⁻¹ and nucleic acid in the 1540-1645 cm⁻¹ band have differences which can be identified using the boosted tree classification algorithm 110.

As mentioned above, the spectral information at least partially or fully available in the captured Raman spectra was analyzed using the boosted tree classification algorithm 110 which determines classification criteria allowing real-time assessment of the cancer status associated with each captured Raman spectrum, Using the boosted tree classification algorithm 110, distinguishing normal brain from tissue with the presence of cancer cells (including both invasive and dense cancers) with an accuracy of 92%, sensitivity of 93% and specificity of 91% was achieved with the system 100, as detailed in Table 2.

Table 2 presented herebelow shows a comparison of tissue classification based on Raman spectroscopy using the system 100 with histopathology, calegorized by grade of glioma or tissue type. The “clinical practice” category indicates the performance neurosurgeon's assessment (from visual inspection and MRI). All measurements on normal brain (n=66 tissue samples; see Table 1) were used in calculating specificity, because it is not related to grade or type. A two-sided normal-based 95% confidence interval (CI) of less than ±5% was obtained for each category.

TABLE 2
Cancer status assessment with the system 100 compared to hispathology

| | Accuracy | Sensitivity | Specificity |
|---|---|---|---|
| | (%) | (%) | (%) |
| WHO grade | | | |
| 2 | 91 | 91 | 91 |
| 3 | 91 | 89 | 91 |
| 4 | 93 | 94 | 91 |
| Tissue type | | | |
| Dense cancer | 93 | 97 | 91 |
| invasive | 90 | 89 | 91 |
| cancer cells | | | |
| Total | 92 | 93 | 91 |
| Clinical practice | 73 | 67 | 86 |

Equations (1) to (3) as set out below were used to calculate the accuracy, the sensitivity and the specificity, respective;

Accuracy=TP+TN(TP+TN+FP+FN) (1);

Sensitivity=TP/(TP+FN) (2); and

Specificity=TN/(FP+TN) (3);

wherein TP is the number of true positives, TN is the number of true negatives, FP is the number of false positives and FN is the number of false positives.

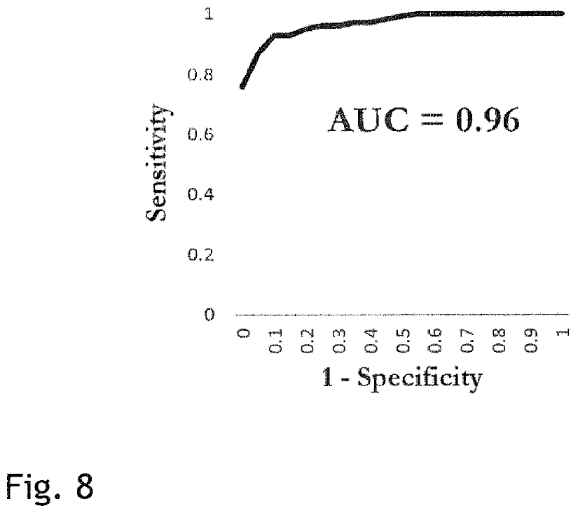

FIG. 8 shows a receiver operating characteristic (ROC) curve obtained from the assessment of the cancer status of the biological tissue measured in this example experiment. The ROC curve shows an ordinate axis associated with sensitivity (or with true positive rate) and an abscissa axis associated with fall-out (or false positive rate) which is calculated as 1—Specificity. The ROC curve has an area under the curve (AUC) of 0.96 for cancer status assessment of all samples with cancer cells (from all grades of glioma and including both dense and invasive cancers). In comparison, the sample labels (either normal brain or cancer) given by the surgeon after visual inspection using a bright-field microscope and MR guidance produced an accuracy of 73%, a sensitivity of 67% and a specificity of 86%. As reported in Table 2, using the captured Raman spectra, cancer status assessment accuracies of 90% or more between normal brain and all tumor grades, as well as between normal brain and either dense cancer or the invasive cancer cell categories was enabled. Distinguishing WHO grade 2 from grade 3 and 4 gliomas in the dense cancer population with an accuracy of 82% using the system 100 was possible. However, distinguishing WHO grades in the normal brain infiltrated with invasive cancer cells or between grade 3 and grade 4 gliomas was found more challenging.

To estimate a cancer cell density threshold that can be detected by the system 100, a histological cell counting was performed for a subset (n=14) of the 56 samples designated as normal brain infiltrated by invasive cancer cells (as seen in Table 2). The 14 samples were selected because they were determined by the pathologist to correspond (on the basis of the analysis of all H&E images) to those with the lowest density of cancer cells. Of these 14 samples, 5 were false negatives using the system 100. A false negative refers to when a tissue is assessed to be normal while cancer cells were found in the corresponding H&E-stained biopsy samples. The remaining nine samples were true positive, as assessed with the system 100.

For each of the 14 samples, multiple regions of interest (each 250 μm×250 μm) were delineated by the neuropathologist on the digitally scanned H&E images. The total number of normal and cancer cells was determined, and the average over the multiple regions of interest was established for cell count per area. The cancer cell counting was validated with mutant IDH1 (R132H) immunihistochemistry. The cancer cell count per area, the total cell per area, and the cancer cell burden (cancer cell count, divided by the total cell count) determined by H&E are reported in Table 3. All false-negative Raman spectroscopy classifications corresponded to <15% cancer cull burden, and all samples having tested positive for cancer (based on spectroscopy) had >15% cancer cell burden. In absolute terms, the system 100 was able to detect the presence of as few as 17 human cancer cells per 0.062.5 mm². These findings are important because minimizing the volume of residual cancer has a measurable impact on the patient's survival.

Table 3 presented herebelow shows the cancer cell resolution capability of the system 100, in accordance with this example experiment. The total number of cells (both normal and cancer cells) and the number of cancer cells were quantified in 14 different patient samples of normal brain invaded with cancer cells. Cells were counted in multiple areas of 250 μm×250 μm (0.0625 mm²), and the average was determined. Samples with an asterisk (*) are those for which cancer cell density was quantified using both H&E and IDH1 (R132H) immunihistochemistry (IHC). These samples have the cell count values obtained using IHC in parentheses. Note that 1 of the 14 samples had both gray matter and white matter, explaining why cell counting information is presented for both in that case. Each of the other samples was either all gray matter or all white matter.

TABLE 3
Estimating the cancer cell resolution capability of the system 100

| | Raman | | | |
|---|---|---|---|---|
| | classification | | | |
| | (positive or | Total celt | Cancer cell | Cancer cell |
| Biopsy | negative for | count per | count per | burden |
| sample | cancer cells) | area | area | (%) |
| 1 | Positive | 95 | 17 | 18 |
| 2 | Positive* | 104 (IHC: 70) | 30 (IHC: 19) | 29 (IHC: 27) |
| 3 | Positive | 78 | 55 | 71 |
| 4 | Positive | 85 | 58 | 69 |
| 5 | Positive | 113 | 98 | 87 |
| 6 | Positive | 92 | 83 | 90 |
| 7 | Positive | 65 | 52 | 80 |
| 8 | Positive* | 78 (ICH: 74) | 65 (IHC: 59) | 85 (IHC: 80) |
| 9 | Positive | 49 | 25 | 51 |
| | | (gray matter) | (gray matter) | (gray matter) |
| | | 74 | 60 | 81 |
| | | (white matter) | (white matter) | (white matter) |
| 10 | Negative | 28 | 2 | 8 |
| 11 | Negative* | 35 (IHC: 51) | 4 (IHC: 6) | 11 (IHC: 12) |
| 12 | Negative | 43 | 5 | 12 |
| 13 | Negative | 56 | 6 | 11 |
| 14 | Negative* | 136 (IHC: | 174 (IHC: 10) | 13 (IHC: 9) |
| | | 118) | | |

In the detailed example experiment set out above, it was shown that intraoperative Raman spectroscopy is well suited for accurate, sensitive and specific tissue assessment and classification of invasive brain cancers for grade 2 to 4 gliomas.

### Description of an Alternate Embodiment of the System 100

According to the challenges disclosed hereinabove, cancer tissue can often be difficult to distinguish from healthy tissue during surgery. Residual invasive brain cancer cells following surgery are the source of recurrence, and residual brain cancer cells negatively affect patient survival. To our knowledge, preoperative or intraoperative technology to identify all brain cancer cells that have invaded the normal brain would be useful.

Gliomas are one of the most fatal tumor types, and constitute 80% of all malignant brain tumors. Brain cancers such as grade 2 and 3 astrocytomas, oligodendrogliomas, and GMBs locally invade into the normal brain, resulting in a decreasing gradient of cancer cells that extend from the main cancer mass into the normal brain. The standard treatment for brain cancer is to remove the tumor surgically, which is largely guided by visual inspection, followed by radiation and chemotherapy.

Bright field macroscopic detection of this decreasing gradient has been found difficult. Magnetic resonance imaging (MRI) or X-ray computed tomography (CT), which serves as a preoperative (pMRI, pCT), and occasionally intraoperative (iMRI, iCT), guide to surgery is also unable to detect the full extent of this cellular invasion and suffers from registration issues due to brain shift. This inability to fully visualize invasive brain cancers directly results in incomplete surgical resections, and in the absence of effective adjuvant therapies, negatively impacts survival.

The median overall survival period of patients suffering from GBM is only 14.6 months. In high-grade gliomas treated with surgical resection, 80% of tumor recurrences originate from remnants of the tumor left by resection. A smaller volume of residual tumor results in improved patient prognosis. Complete resection is the most significant factor in reducing recurrence rate and improving patient survival. This can be true for low-grade gliomas, where long-term studies have shown significant improvements in patient survival after gross total resection. Conversely, the removal of healthy tissue can cause serious issues with cognitive functions such as speech, memory, vision, and balance. A goal of having no residual cancer cells, while not removing excess healthy tissue, represents a significant challenge in brain tumor resection. Taking advantage of the molecular signatures of gliomas is possible through the use of sub-millimeter single-point Raman spectroscopy, as disclosed herein, to guide tumor removal during surgical resection. in particular, infiltrative regions which frequently do not show up on MR or CT images can cause residual tumor to be left after surgery, leading to recurrence.

Intraoperative navigation technology may be an insufficient guide to surgery because it is based on preoperative MRI that does not adequately delineate subtle tumor infiltrations or low-grade disease. Methods based on MRI tend to not reveal the full extent of tumors end spatial registration errors due to tissue deformation lead to inaccurate resections.

Fluorescence-guided surgery with 5-aminolevulinic acid is a newer approach to guide for GBM surgery and has been shown to be more sensitive than MRI at detecting brain cancer cells, improving the extent of resection in certain situations and extending survival.

Despite the significant survival benefits of complete resection in low-grade gliomas, and advances in fluorescence-guided surgery, studies describing its role in low-grade disease are limited. It has not been shown to be capable of accurately detecting rare infiltrative GBM calls and is not able to adequately detect grade 2 and 3 gnomes. It also requires a contrast agent which complicates clinical translation, and the targeted drug delivery can be difficult in the brain.

The integration of Raman, fluorescence and reflectance spectroscopy for brain tumor surgery is proposed herein. Furthermore, Raman spectroscopy has not been used for in vivo detection of invasive cancer cell populations in humans during surgery.

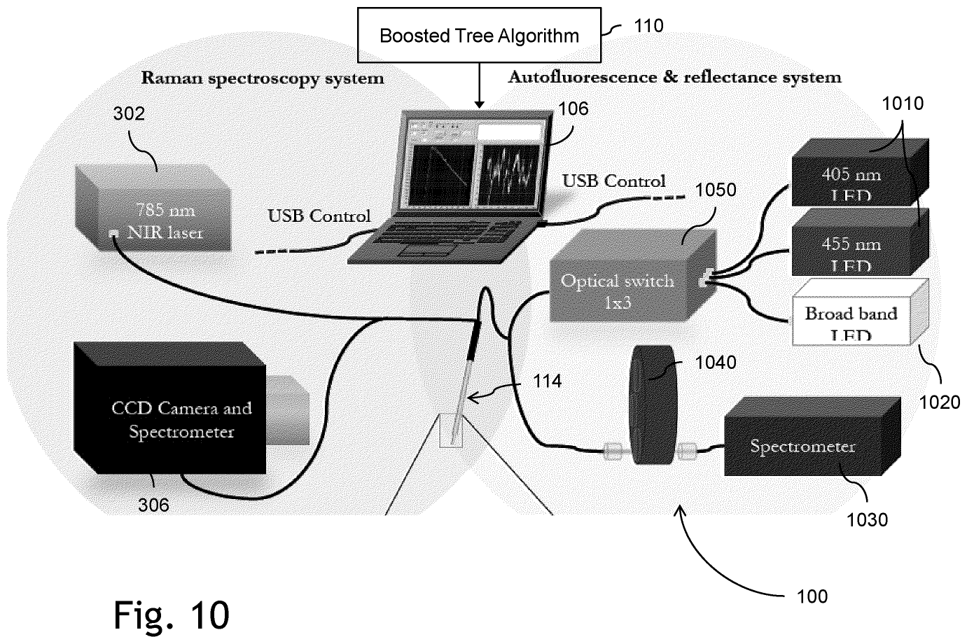

FIG. 10 shows a schematic view of the system 100 which involves a combination of three complementary techniques: inelastic Raman scattering (RS), fluorescence spectroscopy (FS) and diffuse reflectance spectroscopy (DRS), in accordance with an embodiment. The system 100 is combined with the boosted tree classification algorithm 110 to assess cancer cell populations during surgical resection according to tissue type and grade.

The system 100 comprises at least one fiber-optic probe 114 optically coupled to a Raman spectroscopy source for emission of Raman spectroscopy light at 785 nm; the fiber-optic probe 114 is also optically coupled to the spectrometer 306 for Raman spectroscopy light collection; to at least one fluorescence excitation source 1010; to at least one diffuse reflectance source 1020 for emission of excitation light for fluorescence and/or diffuse reflectance; and to a FS/DR spectrometer 1030 for collection of light for measurement of diffuse reflectance and/or fluorescence, using an appropriate tunable filter 1040. In this embodiment, the fiber-optic probe 114 can be coupled to optical components 1010 and 1020 via an optical switch device 1050.

In another embodiment, the system 100 combines inelastic Raman scattering (RS) spectroscopy with at least one of fluorescence spectroscopy (FS) or diffuse reflectance spectroscopy (DRS). The system 100 can include a system interface for communicating with the computer system 106. The system 100 can comprise classification algorithms for the classification of tissue samples according at least to the tissue type and oracle. Further, the classification algorithm can be the boosted tree classification algorithm. Indeed, using the system 100, there is disclosed a method of detection of brain cancer cells comprising the analysis of data obtained from inelastic Raman scattering (RS) spectroscopy and at least one of fluorescence spectroscopy (FS) or diffuse reflectance spectroscopy (DRS). Further, the disclosed method can comprise calibration of data, referencing of data, collection of data from the Raman spectroscopy system 102, data processing, obtaining results, saving results and displaying the results to a user.

In an embodiment, the system 100 relates to an intraoperative device, system and method to detect brain cancer cells, characterize brain tumors during surgery based on one or on several biomarkers of disease. In an embodiment, the system 100 is used for the measurement of inelastic scattering spectra using Raman spectroscopy comprising a fiber-optic probe 114 for light delivery and collection, a system interface, a near-infrared laser 302 used for Raman spectroscopy right excitation, a spectrograph combined with a charge-coupled device spectroscopy detector 305, as described hereinabove. In an embodiment, the system 100 further comprises at least one other spectrometer 1030 for the detection of diffuse reflectance and/or fluorescence spectroscopy, In another embodiment, the system 100 comprises classification algorithms, including but not limited to boosted trees methods, for the classification of tissue samples according at least to the tissue type and grade. In another embodiment, notably for applications related to neurosurgery, the system 100 can further be connected to a neuronavigation system such as the one shown at 310. Pre-operative or intraoperative structural medical images such as pMRI, iMRI, pCT, or iCT, are spatially-registered with the captured Raman spectroscopy spectra. In another embodiment, the fiber-optic prone 114 used for light delivery and collection is able to collect spectroscopy measurements. In an embodiment, the fiber-optic probe 114 is configured so that it can be used for multi-spectroscopy measurements. In another embodiment, the fiber-optic device 114 is configured so that it can additionally measure diffuse reflectance and fluorescence spectroscopy. In another embodiment, the system interface is used to control one or more components of the system 100. The system interface is composed of a material layer (control or equipment and data acquisition), a processing layer (algorithm and data processing) and an interaction layer (result display to the user). It implements the data processing method described as another embodiment of this disclosure. Another embodiment of the system 100 uses a LabVIEW™ interface configured to handle control of the laser sources 302, 1010 and 1020 and spectrometers 306 and 1030, and manage data acquisition.

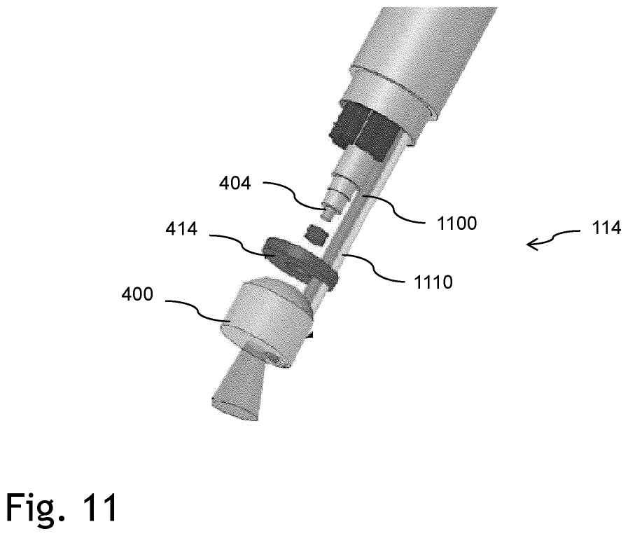

FIG. 11 shows a schematic view of an example of the fiber-optic probe 114. In addition to the elements described hereabove with reference to FIG. 4, the fiber-optic probe shown in FIG. 11 has an RS collection optical-fiber 1100 and a DRS collection optical-fiber 1110 for collecting an RS spectroscopy response and a DRS spectroscopy response, respectively. It can be seen in FIG. 11 that neither collection optical-fibers 1100 and 1110 are filtered by the LP filter 414 and extends through both the LP filter 414 and the interrogation lens 400. The RS and DRS excitation light can be provided to the biological tissue by the excitation optical-fiber 404. In another embodiment, different configurations of the RS and DRS excitation and collection optical-fibers can be found appropriate.

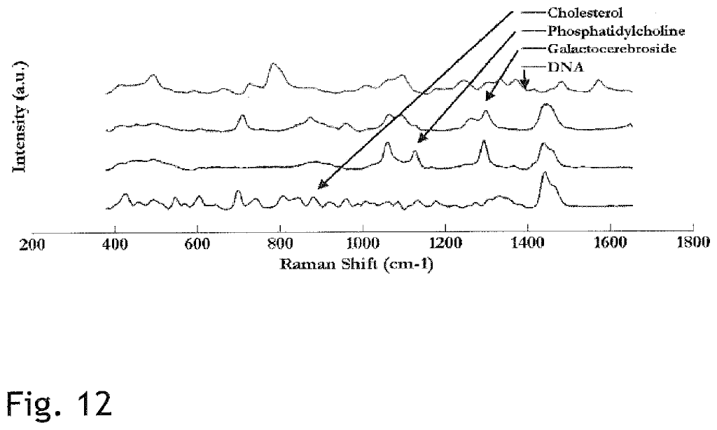

FIG. 12 is a graph showing Raman spectra associated with Raman spectroscopy responses of different molecules. As depicted, Raman spectra associated with a cholesterol molecule, a phophatidylcholine molecule, a galactocerebroside molecule and a DNA molecule are shown, in accordance with an embodiment.

In another embodiment, the fiber-optic device 114 used for light delivery and collection allows for intraoperative measurement of the inelastic scattering Raman spectra, These spectra represent molecular components in the interrogated tissue 104. In some embodiments of the system 100, the molecular signatures measured by Raman Spectroscopy can be used to properly identify invasive tumor tissue which are not easy to distinguish by visual inspection, intraoperative MR-guidance, preoperative MR-guidance, preoperative CT-guidance, or intraoperative CT-guidance. In some embodiments, the fiber-optic probe 114 is provided in the form of a hand-held probe.

The method to process data can stem from the measurements performed on the brain and data resulting, from classification algorithms, to identify the tissue type and WHO grade of the sample tested. The method described herein has a sequence of steps which includes: calibrating data, collecting data using a fiber-optic probe 114, processing the collected data to obtain results, saving the results and displaying the results, for instance. In an embodiment, a step of the method is calibrating the data obtained from the measurements performed on the brain. This step can include background reference measurements of ambient and other light sources, and for some embodiments, calibration measurements of a silicon sample or a Tylenol™ sample. Another step of the method is collecting data of optical interactions with the biological tissue, including any combination of Raman scattering, diffuse reflectance, and fluoresce spectroscopy. This data is acquired from the spectrometer(s) 306 and 1030 which are connected to the fiber-optic probe 114. Another step of the method is processing the acquired data. This step can include measurement averaging to reduce noise, background subtraction, normalization by laser power, and normalization by intrinsic fluorescence in accordance with a fourth-order polynomial, for instance.

As mentioned above, calibrating the fiber-optic probe 114 can allow for comparing Raman spectra measured with different fiber-optic probes 114, which can be useful in practice. Indeed, once a given fiber-optic probe 114 is properly calibrated, the Raman spectra captured with the given fiber-optic probe are generally exempt from artifacts associated with a response function of the given fiber-optic probe 114. Therefore, Raman spectra captured with the given fiber-optic probe can be compared to Raman spectra that would be captured with another fiber-optic probe, for instance. In an embodiment, the set of reference Raman spectra used for determining the reference data are captured using different calibrated fiber-optic probes 114.

In an embodiment, the method includes performing a principal component analysis (PCA) on all Raman spectra data to separate samples, In another embodiment, the method involves use of supervised learning analysis, including but not limited to support vector machines and boosted trees methods for the classification of tissues in samples. In another embodiment, the method has a step of using chemometrics analysis to extract the molecular information associated with normal, infiltrative and cancer tissue. Another step of the method may be saving and displaying results on a display such as a screen in the field of view of a surgeon during a live surgery, for instance.

In the embodiment illustrated in FIG. 7, the system 100 includes the fiber-optic probe 114 for simultaneous use with three types of spectroscopy. The multi-spectroscopy probe 114 is a functional part of the system 100 that is by the neurosurgeon in the sterile section of the operating room. The hand-herd probe 114 used is small, and operates in real-time, making it extremely convenient for intraoperative use during brain tumor resection. It is connected with the other apparatus by one or more fiber-optic cable(s). A LabVIEW™ interface is used to control each component of the system 100. The 785 nm spectrum stabilized near-infrared (NIR) laser 302 (e.g. Innovative Photonic Solutions, N.J., USA) is used for Raman spectroscopy light excitation, and the collection cable of the probe is connected with a spectrograph combined with a high-resolution charge-coupled-device (CCD) spectroscopy detector 306 (e.g. ANDOR Technology, Belfast, UK). The probe 114 can be used to study undistorted diffuse reflection and fluorescence. An emission cable is used to deliver light for fluorescence or diffuse reflectance, while another cable is used for collection. Different light sources 1010 can be chosen for reflectance and fluorescence (LED, Thorlabs, N.J., USA) and another spectrometer 1030 is used in this embodiment for this part he system 100 (Ocean Optics, Fla., USA).

As mentioned above, an example experiment, using the exemplary embodiment of the system 100 and method described herein, investigated the use of Raman Spectroscopy for intraoperative use in 17 adult neurosurgical patients at the Montreal Neurological Institute and Hospital with grade 2-4 gliomas. Patients were selected based on suitability for undergoing craniotomy for tumor resection and the ability to collect intact heterogeneous brain tissue samples containing normal and malignant tissue. Exclusion criteria included neurological status and type of craniotomy procedure. Patients received a complete preoperative neurological examination, and standard clinical imaging, cognitive neuropsychological tests and BOLD fMRI-DTI. During surgical resection, a hand-held fiber-optic probe 114 was used to measure the Raman signal of in vivo tissue samples (FIG. 4 and FIG. 5). Between 5 and 15 measurements were taken for each patient.

In this embodiment, the hand-held probe 114 has fiber optic cables (e.g. EmVision, LLC) connected to a near-infrared (NIR) spectrum stabilized laser source 302 emitting at 785 nm (innovative Photonic Solutions). The hand-held probe 114 is also connected to a high-resolution CCD spectroscopic detector 306 (e.g. ANDOR Technology). The laser 302 and the CCD 300 are connected to a personal computer (PC) 106 with a LabVIEW™ interface, to obtain the Raman spectra and visualize in real-time. All data processing was performed in MATLAB™ (Mathworks, Inc.). The hand-held probe 114 has the identifiable markers 501 for spatial registration with the CAS 310 (e.g. Medtronic™ StealthStation™ system). The identifiable markers 501 of the CAS system 310 allow for intraoperative guidance of measurement interrogation sites with respect to MRI, an example of which is shown at FIG. 6E.

A variety of classification algorithms have been used to analyze Raman spectra in previous studies, including support vector machines, linear discriminant analysis, and artificial neural networks. The boosted trees algorithm was chosen for analysis based on comparisons of learning algorithms, with superior performance overall. As mentioned above, it is robust to noise in the training data as well as the test date, a quality which is a factor to consider given the rarity of the Raman Effect relative to background signal. Furthermore it may not make assumptions about feature independence, and performs consistently regardless of spectral density. The boosted trees algorithm operates by constructing an ensemble of decision trees from training data. Each decision tree has a classification rule, and operates on the residual of the classification determined by the previous decision tree. Classification was applied using a cross-validation approach. Each spectra from the set of Raman spectra was in turn considered to be the test or reference dote. For each test data, the rest of the spectra were used as training data to train a boosted tree classifier. Cross-validation analysis was also used to determine the optimal number of decision trees for use in the classification, resulting in the use of preferably eight decision trees, for instance. This optimization of the number of trees is to avoid over-fitting the data, while maintaining sufficient complexity for proper assessment.

As mentioned above, Raman spectroscopy was used on 17 patients with grade 2-4 gliomas to determine the ability to accurately identity tumor tissue. Between 5 and 15 measurements were taken per patient, for a total of 161 measurements used. Patient histology information is listed in Table 1, including tumor grade and type. Three classes were used to label tissue normal (not positive for any tumor cells), infiltrated (rare tumor cells present), and tumor (all tumor), Tumor-infiltrated areas are often difficult to identify by visual inspection and do not show up on preoperative MRI, as illustrated schematically in FIGS. 6A and 6D. The majority of patients were confirmed by pathology to have tumor-infiltrated tissue beyond the boundary defined by MRI, See insets 634, 633, and 638 for crosshairs views of MRI region 601 shown in FIG. 6D and insets 640, 642, and 644 for samples of histological tissue images for each tissue type. In an embodiment, the hand-held probe 114 was used to detect the Raman spectra at various locations in and around the tumor for each patient, with an emphasis on locations with rare infiltrative cancer cells.

Referring hack to FIG. 7, averaged spectra of normal and tumorous tissue measurements are shown. These spectra show differences in the molecular signature of the sampled brain tissue. The regions in the spectra which show the most consistent differences between normal and tumor/infiltrated tissue are indicated. Raman spectroscopy provides particular biological information which can be used diaonostically based on the molecular differences of tumor tissue. Tissue with tumor cells shows a decrease in the lipid bands at 700 cm⁻¹ and 1142 cm⁻¹ compared to normal brain, corresponding to cholesterol and phospholipids. The presence of tumor cells also showed an increase in the size of the bands from 1540 cm⁻¹ to 1645 cm⁻¹, corresponding to a higher nucleic acid content than normal brain tissue, as observed previously far GBM. Tumor tissue shows an increase in the 1005 cm⁻¹ band, associated with the breathing mode of phenylalanine in proteins.

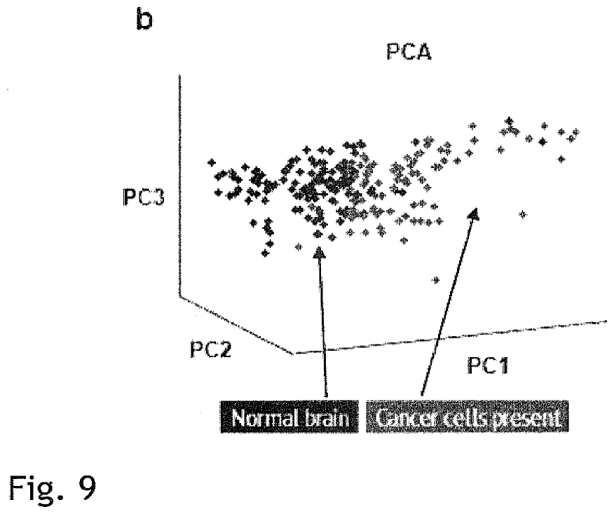

FIG. 9 shows a principal component analysis which illustrates the ability to separate samples based on difference information in the Raman spectra, in accordance with an embodiment.

To utilize all of the spectral information available in the Raman signal, the boosted trees machine learning algorithm was used to analyze the spectra and determine classification criteria for the different tissue categories. As mentioned above, Table 2 shows the classification accuracy for each grade of glioma, as well as for pure tumor and rare infiltrative tumor tissue. The classification results yield an accuracy of 92%, sensitivity of 93 and specificity of 91%. In comparison, the sample labels given by visual inspection and MR-guidance produced an accuracy of 73%, sensitivity of 37%, and specificity of 86%.

It is demonstrated that this technique can detect ail cell populations within grade 2-4 gliomas, including the previously undetectable diffuseiy invasive cells. It accurately differentiates normal brain from dense cancer and rare invasive cancer cells (accuracy=92%, sensitivity=93%, specificity=91%). The results indicate that this technique is sensitive and specific to glioma tumor tissue.

Both the sensitivity and specificity show significant improvement over the values representing visual assessment and MR-guidance for the neurosurgeon. The robustness of the method to grade is advantageous to reducing the chance of recurrence among all glioma patients and improving patient survival for all malignant glioma tumors. A sensitivity of 91% in grade 2 gliomas, and 89% in rare tumor infiltration was obtained, which is beyond what has been achieved by other technologies such as fluorescence-guided surgery.

Although the embodiment disclosed herein relates to brain cancer cells detection, it will be apparent to someone skilled in the art that other embodiments of the methods and systems described herein can be extended to other types of cancer such as breast, cervix, mouth and throat, to name only a few.

The above description is meant to be exemplary only, and one skilled in the art will recognize that changes may be made to the embodiments described without departing from the scope of the invention disclosed. Modifications which fall within the scope of the present invention will be apparent to those skilled in the art, in light of a review of this disclosure, and such modifications are intended to fall within the appended claims.

What is claimed is:

1. A method for assessing a cancer status of biological tissue in real time, the method comprising the steps of:
acquiring a Raman spectroscopy response of biological tissue in less than about 0.05 sec using a fiber-optic probe of a fiber-optic Raman spectroscopy system, the probe comprising an excitation optical fiber for delivering Raman excitation light generated by a Raman spectroscopy source to an interrogation lens at an interrogation tip; collection optical fibers for collecting Raman spectroscopy response from biological tissue; a band pass filter at the interrogation tip to filter the Raman excitation light to interrogate the biological tissue at a specified wavelength; and a long pass filter co-located with the band pass filter to filter out the Raman excitation light from the Raman spectroscopy response collected by the collection optical fibers;
generating a Raman spectrum indicative of the Raman spectroscopy response;
inputting the Raman spectrum into a classification algorithm of a computer program, wherein the classification algorithm is configured to permit an analysis of the Raman spectrum in its entirety and to provide a robustness towards noise in the generated Raman spectrum so as to enable the acquisition of the Raman spectroscopy response of the biological tissue in less than about 0.05 sec;
using the classification algorithm for comparing, in less than one second, the Raman spectrum in its entirety to reference data and assessing the cancer status of the biological tissue based on said comparison, the reference data being previously determined based on a set of reference Raman spectra indicating Raman spectroscopy responses of reference biological tissues wherein each of the reference biological tissues is associated with a known cancer status; and
generating a real-time output indicating the assessed cancer status of the biological tissue.

2. The method of claim 1, wherein the method is conducted intraoperatively, and the step of obtaining the Raman spectrum includes intraoperatively obtaining the Raman spectrum from the biological tissue in vivo, and the step of generating includes intraoperatively generating the real-time output.

3. The method of claim 2, wherein the reference data is preoperatively determined by conducting a training process of the classification algorithm using the set of reference Raman spectra.

4. The method of claim 1, wherein the step of using the classification algorithm further comprises determining classification criteria for each one of a plurality of decision trees of the classification algorithm based on the reference data.

5. The method of claim 4, wherein the step of using the classification algorithm further comprises determining an optimal number of decision trees.

6. The method of claim 5, further comprising selecting the number of decision trees to be eight.

7. The method of claim 1, wherein the classification algorithm comprises a boosted tree algorithm.

8. The method of claim 1, further comprising obtaining at least one signal characteristic representative of the biological tissue and inputting said at least one signal characteristic into the classification algorithm, said at least one signal characteristic including at least one of diffuse reflectance spectroscopy and fluorescence spectroscopy.

9. The method of claim 8, further comprising using the fiber-optic probe to capture said at least one signal characteristic.

10. The method of claim 1, wherein the biological tissue is brain tissue and the method includes intraoperatively assessing the cancer status of the brain tissue during neurosurgery.

11. A system for assessing a cancer status of biological tissue, the system comprising;
a fiber-optic Raman spectroscopy system including a fiber-optic probe comprising an excitation optical fiber for delivering Raman excitation light generated by a Raman spectroscopy source to an interrogation lens at an interrogation tip; collection optical fibers for collecting Raman spectroscopy response from biological tissue; a band pass filter at the interrogation tip to filter the Raman excitation light to interrogate the biological tissue at a specified wavelength; and a long pass filter co-located with the band pass filter to filter out the Raman excitation light from the Raman spectroscopy response collected by the collection optical fibers, the fiber-optic Raman spectroscopy system being configured to acquire a Raman spectroscopy response of biological tissue in less than about 0.05 sec and to generate a Raman spectrum indicative of the Raman spectroscopy response after interrogating the biological tissue in real-time with the fiber-optic probe; and
a computer comprising a processor coupled with a computer-readable memory, the computer-readable memory being configured for storing the Raman spectrum and computer executable instructions that, when executed by the processor, perform the steps of:
using a classification algorithm for intraoperatively comparing, in less than one second, the Raman spectrum in its entirety to reference data, and assessing the cancer status of the biological tissue based on said comparison, the reference data being previously determined based on a set of reference Raman spectra indicating Raman spectroscopy responses of reference biological tissues wherein each of the reference biological tissues is associated with a known cancer status, wherein the classification algorithm is configured to permit an analysis of the Raman spectrum in its entirety and to provide a robustness towards noise in the generated Raman spectrum so as to enable the acquisition of the Raman spectroscopy response of the biological tissue in less than about 0.05 sec; and
generating a real-time output indicating the cancer status of the biological tissue.

12. The system of claim 11, wherein the system is used intraoperatively, and the step of generating the real-time output performed by the computer executable instructions includes intraoperatively generating the real-time output, the real- time output including at least one of a visual and an audible signal indicative of the cancer status of the biological tissue.

13. The system of claim 11, wherein the fiber-optic probe is hand-held.

14. The system of claim 11, wherein the computer executable instructions, when executed by the processor, further perform the step of: determining classification criteria for each one of a plurality of decision trees of the classification algorithm based on the reference data.

15. The system of claim 14, wherein the computer executable instructions, when executed by the processor, further perform the step of selecting an optimal number of the decision trees.

16. The system of claim 11, wherein the reference data is preoperatively determined in a training process of the classification algorithm using the set of reference Raman spectra.

17. The system of claim 11, wherein the classification algorithm comprises a boosted tree algorithm.

18. The system of claim 11, further comprising at least one of a fiber-optic diffuse reflectance spectroscopy system and a fiber-optic fluorescence spectroscopy system, wherein the diffuse reflectance spectroscopy system generates at least one diffuse reflectance spectrum indicative of a diffuse reflectance spectroscopy response of the biological tissue, and the fluorescence spectroscopy system generates at least one fluorescence spectrum indicative of a fluorescence spectroscopy response of the biological tissue.

19. The system of claim 18, wherein the computer executable instructions, when executed by the processor, further perform the step of: using at least one signal characteristic into the classification algorithm, said at least one signal characteristic including at least one of the diffuse reflectance spectroscopy spectrum and fluorescence spectroscopy spectrum.

20. The system of claim 19, wherein the fiber-optic probe is configured to capture at least one of the diffuse reflectance spectroscopy response and the fluorescence spectroscopy response.

21. The system of claim 11, wherein the biological tissue is brain tissue, and the system is operable to intraoperatively assess the cancer status of the brain tissue during neurosurgery.
